(19) United States
(12) Patent Application Publication
(10) Pub. No.: US 2006/0019892 A1
(43) Pub. Date: Jan. 26, 2006

(54) Conopeptides and methods of use

(75) Inventor: Frank Mari, Boca Raton, FL (US)

Correspondence Address: AKERMAN SENTERFITT, P.O. BOX 3188, WEST PALM BEACH, FL, 33402-3188, US

(73) Assignee: Florida Atlantic University, Boca Raton, FL (US)

(21) Appl. No.: 11/149,757

(22) Filed: Jun. 10, 2005

Related U.S. Application Data
(63) Continuation-in-part of application No. 10/794,640, filed on Mar. 5, 2004.
(60) Provisional application No. 60/452,030, filed on Mar. 5, 2003.

(51) Int. Cl. A61K 38/16; C07K 14/435

(52) U.S. Cl. 514/12; 530/324

(57) ABSTRACT

Isolation, synthesis and therapeutic use of compounds and related compositions based on a new class of conopeptides comprising the modified amino acid γ-hydroxyvaline (Hyv=V*). These isolated peptides are the first known example of a naturally occurring polypeptide chain containing Hyv. The active peptides, termed γ-Hydroxyconophans are heavily hydroxylated small peptides. These peptides contain a definitive structural motif which is a double modification of the polypeptide chain (γ-OH-Hyv-D-Trp).

### CROSS REFERENCE TO RELATED APPLICATIONS

The present application is a Continuation-In-Part of U.S. patent application U.S. Ser. No. 10/794,640, now pending, which claims the priority of U.S. provisional patent application No. 60/452,030, entitled “Therapeutic Agents Containing Gamma Hydroxylated Amino Acids,” filed Mar. 5, 2003. The foregoing is incorporated herein by reference in its entirety.

### STATEMENT AS TO FEDERALLY SPONSORED RESEARCH

This invention was made with U.S. government support under grant numbers GM066004 and CA77402 awarded by the National Institutes of Health. The U.S. government may have certain rights in the invention.

### FIELD OF THE INVENTION

This invention relates generally to the fields of medicine and neuropharmacology. More particularly, the invention relates to a new class of conopeptide compounds useful for binding with specificity to cellular targets such as cell surface receptors.

### BACKGROUND

The venom of predatory marine mollusks belonging to the genus Conus (cone snails) is a rich source of compounds of demonstrated and potential therapeutic use. The Conus venom is a complex mixture of peptides (conopeptides) that elicit a wide range of neurophysiological responses. The venom is used by this marine organism to paralyze its prey. Venom is produced and released by means of a complex venom apparatus including a tubular venom duct wherein the venom is produced by epithelial cells and a muscular bulb used to propel the venom. The bulb is connected to a specialized radular tooth that is used as a harpoon to both impale the prey and reel it in.

Several conopeptides have been shown to be valuable therapeutic agents for the treatment of a variety of neurologically related conditions. The polypeptide components of the venom are produced on ribosomes as protein precursors that subsequently undergo post-translational modifications. The active conopeptides are produced by proteolytic cleavage of the protein precursors. Several classes of conopeptides have been described which exhibit exquisite specificity towards specific neuronal targets such as numerous subclasses of acetylcholine receptors, subtypes of ion channels including sodium, potassium and calcium channels, glutamate receptors, N-Methyl-D-Aspartate (NMDA) receptors and neurotensin receptors. A calcium channel blocking drug based on \acute{ω}-conotoxin MVIIA is presently on the market as a treatment for chronic pain. Other conopeptide-based compounds are thought to be useful in the treatment of Alzheimer's disease, immune system regulation, control of angiogenesis and arrest of malignant growth (Tsetlin, V. I. and Hucho, B., FEBS Lett. 557:9-13, 2004).

There exists a vast number of known species in the genus Conus. Venom from only a fraction of these has been analyzed for the presence of potentially useful conopeptides. Furthermore, the number of distinct conopeptides within any given Conus venom has been shown to be very large. Given the proven usefulness of those few conopeptides presently characterized, there exists great potential to tap this natural combinatorial peptide library as a source for therapeutically useful new compounds.

### SUMMARY

The invention relates to the isolation, synthesis and therapeutic use of compounds and related compositions based on a new class of Conus conopeptides containing the modified amino acid γ-hydroxyvaline (Hyv=V*). These isolated peptides are the first known example of a naturally occurring polypeptide chain containing Hyv. The peptides contain a unique structural motif which is a double modification of the polypeptide chain in contiguous residues, i.e., γ-OH-Hyv-Trp. Yet a further unusual feature of some of the peptides is the fact that the Trp is in the form of D-Trp. The presence of the γ-OH-Hyv-D-Trp motif in the peptides defines a new class of conopeptides designated herein as γ-Hydroxyconophans. γ-Hydroxyconophans may be defined as containing an amino acid sequence of the general formula H₃CC(O)-Hyv-D-Trp-NH₂. In comparison with known classes of conopeptides, the γ-Hydroxyconophans are particularly atypical because (i) they are not three-dimensionally constrained, in marked contrast to most conopeptides; (ii) they have a high content of hydroxylated residues; iii) they can be unusually short, some embodiments being about eight amino acids in length; and (iv) their primary amino acid sequences have no match with other known peptides in any sequence database.

The amino acid sequences of members of this new class of peptides are based on the discovery of unique octapeptide sequences in the venom of predatory cone snails of the species Conus gladiator and Conus mus. Using a combination of high-resolution analytical methods including nano-NMR and tandem mass spectrometry (MS/MS), the primary sequences, post-translational modifications, chirality and three-dimensional structures of several native conopeptides were elucidated.

Peptides designated herein as gla-1/gla-1′ have the backbone amino acid sequence: A-P-A-N-S-V-W-S (SEQ ID NO:2). Those designated mus-1/mus-1′ have the backbone amino acid sequence: A-P-S-N-S-V-W-S (SEQ ID NO:3). Even without consideration of the unusual modifications discovered to be present in the native conopeptides (i.e., γ-hydroxyproline at residue 2, γ-hydroxyvaline at residue 6 and D-tryptophan at residue 7), the primary structures of the octapeptides represented by SEQ ID NOS:2 and 3 do not match any contiguous sequence of eight amino acids in known or hypothetical proteins described in any database.

Although functional studies using compounds based on γ-Hydroxyconophan sequences in in vitro assays demonstrated a modulatory effect of these peptides on flux of Ca⁺⁺ ions in primary cultures of neurons, consistent with selective binding to target receptors on the cell surfaces, it is likely that specific embodiments of the conopeptides will influence the flux of various cellular ions including sodium, potassium, and chlorine. Therapeutic agents based on these novel sequences and modifications of the amino acid residues therein are likely to find use in a wide variety of applications in which cellular receptors are to be targeted for localization and/or modulation of downstream effects mediated by the receptors.

Accordingly, one aspect of the invention includes an isolated γ-Hydroxyconophan peptide. The peptide includes the amino acids γ-OH-Val and D-Trp in contiguous residues. Other embodiments of the peptide include the amino acid sequence shown herein as SEQ ID NO:2. This peptide is based on the primary sequence of native gla-1 peptide discovered and purified from the venom of Conus gladiator. In another embodiment, the peptide includes the amino acid sequence of SEQ ID NO:3. The latter peptide is based on the corresponding primary sequence (SEQ ID NO:3) found in the mus-1/mus-1′ octapeptide discovered and purified from the venom of Conus mus.

Yet other embodiments of the isolated peptides contain modified amino acids and include an amino acid sequence of the following general structure: A-O-X₁-N-S-X₂-W-S (SEQ ID NO:1) wherein:
O is γ-γ-hydroxyproline;
X₁ is A or S;
X₂ is V or γ-hydroxyvaline (V*); and
W is D-Tryptophan.

Preferred embodiments of the A-O-X₁-N-S-X₂-W-S peptides include peptides having the amino acid sequences shown herein as SEQ ID NOS:5-8. The latter sequences correspond, respectively, to those found in native gla-1, native mus-1 (SEQ ID NOS:5 and 6), and their presumed precursors gla-2 and mus-2 (SEQ ID NOS:7 and 8).

The peptides of the invention can be isolated from an animal, such as a species of Conus, or the peptides can be synthesized by man. The invention further provides pharmaceutical compositions including the isolated peptides.

In another aspect, the invention provides a method of modulating the level of an ion within a cell using the peptides of the invention. The method includes the steps of: (a) providing a cell that responds to a peptide that binds to a chemical structure on the surface of the cell by modulating the level of at least one ion within the cell; and (b) contacting the cell with a peptide including the amino acids γ-OH-Val and D-Trp in contiguous residues, wherein the peptide selectively binds to the chemical structure.

In some embodiments, the chemical structure on the surface of the cell is a cell surface receptor. In preferred embodiments, the receptor is of a selected type, including a calcium channel, a sodium channel, a potassium channel, and a chloride channel. In preferred embodiments, the ion can be a calcium ion, a sodium ion, a potassium ion or a choride ion. In some embodiments, the peptide binds to a receptor that is voltage-gated.

In some embodiments, the peptide used in the method includes the amino acid sequence of SEQ ID NO:2 or SEQ ID NO:3. In other embodiments, the peptide includes an amino acid sequence that may be described as A-O-X₁-N-S-X₂-W-S (SEQ ID NO:1) wherein:
O is γ-γ-hydroxyproline;
X₁ is A or S;
X₂ is V or γ-hydroxyvaline (V*); and
W is D-Tryptophan.

In some embodiments, the method may be practiced using octapeptides having amino acid sequences based on native gla and mus peptides, listed herein as SEQ ID NO's: 5-8.

In another preferred embodiment, the peptides comprise Ser-D-γ-OH-Xaa-Trp, wherein Xaa=Asp, Asn, Ser, Thr, Val, Ala, Gly, Leu, Ile, or Pro in the D- or L-configuration.

In another preferred embodiment, an isolated conopeptide comprises modified valine and proline residues wherein the modified residues are hydroxylated. Preferably, the isolated conopeptides comprise a triad motif identified by Ser-D-γ-Hyv-Trp and the valine is in a D chiral configuration.

In another preferred embodiment, at least one D-Val in the conopeptide is hydroxylated, preferably at least two D-Val residues in the conopeptide are hydroxylated, preferably, each D-Val residue is hydroxylated.

In another preferred embodiment, between about 1% up to 100% of valine residues in the conopeptide are hydroxylated.

In one preferred embodiment, the conopeptide is identified by an one of SEQ ID NO's.: 1-54.

In another preferred embodiment, a composition comprises an isolated conopeptide, wherein the isolated conopeptide comprises modified valine and proline residues in a pharmaceutically acceptable carrier. Preferably, the valine and proline residues are modified by hydroxylation.

In another preferred embodiment, the composition comprises a conopeptide comprising a triad motif identified by Ser-D-γ-Hyv-Trp, wherein the valine residue is hydroxylated. Preferably, the conopeptide is identified by an one of SEQ ID NO's.: 36-54.

In another preferred embodiment, an isolated conopeptide comprising any one of SEQ ID NO's.: 9-35.

In another preferred embodiment, a method for inducing analgesia in a mammal which comprises administering a therapeutically effective amount of a conopeptide identified by an one of SEQ ID NO's.: 9-54. The administration comprises using a delivery means selected from the group consisting of a pump, microencapsulation, a continuous release polymer implant, microencapsulation, naked or unencapsulated cell grafts, injection and oral administration. Preferably, the amount of conopeptide administered is between about 0.001 mg/kg to about 250 mg/kg.

In another preferred embodiment, a pharmaceutical composition comprises a therapeutically effective amount of a conopeptide identified by any one of SEQ ID NO's.: 1-54 or a pharmaceutically acceptable salt or solvate thereof and a pharmaceutically acceptable carrier. The composition can also comprise one or more drugs useful in the treatment of pain.

Although methods and materials similar or equivalent to those described herein can be used in the practice or testing of the present invention, suitable methods and materials are described below. All publications, patent applications, patents, and other references mentioned herein are incorporated by reference for the proposition cited.

Other aspects of the invention are discussed infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1A is three graphs showing gla conopeptide isolation from the venom of Conus gladiator. Upper and middle plots show fractionation of venom extract using Size Exclusion HPLC (SE-HPLC). The SE-HPLC elution profiles are shown at λ=220 (top) and 280 nm (middle). The arrow indicates the selected fractions of Trp-containing gla peptides. The lower plot shows further fractionation of the major SE-HPLC peaks by Reversed Phase HPLC (RP-HPLC). Three peaks corresponding to peptides gla-1, gla-1′ and gla-2 are seen.

FIG. 1B is three graphs showing mus conopeptide isolation from the venom of Conus mus. Arrow indicates the selected fractions of Trp-containing mus peptides. Isolation steps are as described for FIG. 1A. By RP-HPLC (lower plot), peptides mus-1, mus-1′ and mus-2 are purified in three peaks.

FIG. 2A is a NMR 1D proton spectrum (upper graph) and the corresponding ¹H-NMR 2D-TOCSY spectrum (lower image) of gla-1 peptide purified from C. gladiator. The 2D-TOCSY data were recorded at 25° C. using a gHX HR-MAS probe. The NMR assignments of the γ-hydroxyvaline (i.e., HN: d 7.99, 8 Hz; αH: 4.45, m; γCH₂.m 3.15; βCH m 1.89, 6.9 Hz; γCH₃: d 0.52, 7.1 Hz) correlate well with reported values for synthetic γ-hydroxyvaline.

FIG. 2B is a NMR 1D proton spectrum (upper graph) and the corresponding 2D-TOCSY spectrum (lower image) of gla-1′ peptide purified from C. gladiator. Data were recorded as described for FIG. 2A.

FIG. 2C. is a graph showing a NMR 2D-TOCSY spectrum of native gla-2 peptide isolated from the venom of C. gladiator. Data were recorded as described for FIG. 2A.

FIG. 3A is a graph showing mass spectrometry (ESI-MS/MS) spectrum of the gla-1 peptide from C. gladiator.

FIG. 3B is a graph showing ESI-MS/MS spectrum of the gla-1′ peptide from C. gladiator.

FIG. 4A is two graphs showing NMR 1D proton spectra of conopeptides mus-1 (upper plot) and mus-1′ (lower plot) purified from C. mus.

FIG. 4B is a graph showing ESI-MS/MS spectrum of the mus-1 peptide from C. mus.

FIG. 4C is a graph showing ESI-MS/MS spectrum of the mus-1′ peptide from C. mus.

FIG. 5 is three graphs showing a comparison of NMR spectra (1D proton spectrum, 0.2-4.5 ppm region, αH & side chains) of native gla-2 conophan (upper) and corresponding synthetic peptides of gla-2 containing either L-Trp (middle) and D-Trp (lower). The arrows in the upper and lower graphs indicate resonances that are dissimilar in synthetic gla-2 containing L-Trp (middle) compared with native gla-2 (upper) or synthetic gla-2 with D-Trp (lower), confirming the chirality assignment of Trp-7 in gla-2.

FIG. 6A is a graph showing ESI-MS/MS of the native gla-2 conophan from C. gladiator. Spectra were recorded using a LCQ-Deca Ion trap instrument.

FIG. 6B is a graph showing ESI-MS/MS of synthetic gla-2 containing L-Trp.

FIG. 6C is a graph showing ESI-MS/MS of synthetic gla-2 containing D-Trp.

FIG. 7A is a graph showing chromatographic characterization of two synthetic analogs of the gla-1 γ-Hydroxyconophan, i.e., gla-1(Hyv6Thr) and gla-1(Hyv6Thr′). The graph shows a RP-HPLC profile of the two analogs using the chromatographic conditions used in FIG. 1. The difference in retention of these analogs is the same as the gla-1/gla-1′ pair.

FIG. 7B is a graph showing ESI-MS/MS spectral characterization of gla-1 synthetic analog gla-1(Hyv6Thr).

FIG. 7C is a graph showing ESI-MS/MS spectral characterization of gla-1 synthetic analog gla-1(Hyv6Thr′). Comparing FIGS. 7B and 7C with FIGS. 3A and 3B (native gla-1 and gla-1′, respectively), it is seen that the ESI-MS/MS spectra of the two analogs of gla-1 have similar fragmentation patterns to those of the native peptides.

FIG. 8 shows the chemical structure of the native gla-1 octapeptide.

FIG. 9 is a drawing depicting a molecular model of a γ-Hydroxyconophan structural motif H₃CC(O)-Hyv-D-Trp-NH₂. This structure illustrates the proximity of the γ-methyl of Hyv to D-Trp. Evidence of this interaction is provided by the strongly shielded chemical shift of this methyl group shown in FIGS. 3 and 4. D-Trp provides steric impediment to the lactonization process (represented by the electron clouds of the side chains of these modified amino acids), thereby providing stability to polypeptide chains that include this structural motif.

FIG. 10A shows a live specimen of C. regius and FIG. 10B shows the corresponding RP-HPLC chromatogram (C-18 Vydac, linear gradient 0-60% H₂O/ACN with 0.1% TFA in 100 min) of its crude venom. C. regius a widespread Western Atlantic worm-hunting species. This specimen was collected off the Florida Keys in 2-meter depth. The C. regius shows another shell variant known C. regius citrinus whose shell is bright orange. This variation has no effect in their venom composition.

FIG. 11A-11E shows SE & RP-HPLC Separation of the Crude Venom of C. regius. FIG. 11A shows the SE separation of C. regius crude venom. FIG. 11B shows the further separation by SE of fraction 06. FIG. 11C. shows the RP analysis of fraction b. FIG. 11D shows the RP clean-up of fraction 13. FIG. 11E shows the RP analysis of fraction C.

FIG. 12A shows 2D-NMR NOESY spectra of 43 nanomoles of native reg6γ recorded at 25° C. using a 1.7 mm insert in a 3 mm gHCN probe on a Varian Inova 500 MHz spectrometer. The mixing time was 200 ms. FIG. 12B. shows the circular dichroism spectra of a 0.1 μM solution of reg6γ in water at 25° C.

### DETAILED DESCRIPTION

The invention encompasses compositions and methods relating to a novel class of biologically active conopeptides, termed “Conophans, and “γ-Hydroxyconophans” which can be obtained by purification from the venom of predatory cone snails of the genus Conus. Alternatively, the compositions can be synthesized. The below described preferred embodiments illustrate various compositions and methods within the invention. Nonetheless, from the description of these embodiments, other aspects of the invention can be made and/or practiced based on the description provided below.

This new family of linear short conopeptides, described herein, have been termed γ-hydroxyconophans. These conopeptides have a high content of hydroxyl residues: Ser, Hyp and the unprecedented presence of D-γ-hydroxyvaline in their sequence. These conopeptides are the first examples of polypeptides chains containing such an intriguing modification. As an example of this new family, four novel conopeptides from the venom of Conus gladiator (gld-V* and gld-V*′) and Conus mus (mus-V* and mus-V*′) were characterized. These conopeptides comprise the doubly modified amino acid D-γ-Hyv. We have also isolated their respective peptidyl precursors that contain D-Val (gld-V and mus-V).

Unless otherwise defined, all technical terms used herein have the same meaning as commonly understood by one of ordinary skill in the art to which this invention belongs.

As used herein, “γ-Hydroxyconophan” is used to describe a new class of conopeptide (i.e., a peptide derived from a Conus species) which is defined as containing an amino acid sequence of the general formula H₃CC(O)-Hyv-D-Trp-NH₂. A “Conophan” is a presumed precursor of a γ-Hydroxyconophan, in which the valine is not hydroxylated, being defined therefore by the general formula H₃CC(O)-V-D-Trp-NH₂.

The conopeptides of the present invention are useful for the treatment of pain or the induction of analgesia. As used herein the term “treating” also includes prophylaxis of pain in a patient or a subject having a tendency to develop such pain, and the amelioration or elimination or the developed pain once it has been established or alleviation of the characteristic symptoms of such pain. As used herein the term “pain” shall refer to all types of pain. Preferably, the term refers to chronic pains, such as neuropathic pain, and post-operative pain, chronic lower back pain, cluster headaches, herpes neuralgia, phantom limb pain, central pain, dental pain, neuropathic pain, opioid-resistant pain, visceral pain, surgical pain, bone injury pain, pain during labor and delivery, pain resulting from burns, including sunburn, post partum pain, migraine, angina pain, and genitourinary tract-related pain including cystitis, the term shall also preferredly refer to nociceptive pain or nociception.

Pharmaceutical compositions containing a compound of the present invention or its pharmaceutically acceptable salts or solvates as the active ingredient can be prepared according to conventional pharmaceutical compounding techniques. See, for example, Remington's Pharmaceutical Sciences, 18th Ed. (1990, Mack Publishing Co., Easton, Pa.).

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

The active agent is preferably administered in an therapeutically effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, a sufficient amount of the compound to treat or alleviate pain or to induce analgesia at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

By “substantially pure” is meant that a conopeptide of the invention has been separated from components which naturally accompany it, or which are generated during its preparation or extraction. Preferably the peptide is at least 90%, more preferably at least 95%, and most preferably at least 99%, by weight, free from the other peptides and molecules with which it is naturally associated. The purity of the peptides can be measured by any appropriate method, for example, column chromatography, polyacrylamide gel electrophoresis, or HPLC analysis.

### Biological Methods

Methods involving conventional and analytical chemistry, molecular biological and cell biological techniques are described herein. Such techniques are generally known in the art and are described in detail in methodology treatises such as Classics in Total Synthesis. Targets, Strategies, Methods, K. C. Nicolaou and E. J. Sorensen, VCH, New York, 1996; The Logic of Chemical Synthesis, E. J. Coney and Xue-Min Cheng, Wiley & Sons, NY, 1989; and NMR of Proteins and Nucleic Acids, Wuthrich, K., Wiley & Sons, New York, 1986. Molecular biological and cell biological methods are described in treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods involving conventional biology and neurobiology and neuropharmacology are also described herein. Such techniques are generally known in the art and are described in methodology treatises such as Sambrook et al., supr. Conopeptides and related toxins are generally reviewed, for example, in Tsetlin, V. I. and Hucho, B., FEBS Lett. 557:9-13, 2004, and in Mari, F. and Fields, G. B, Chimica Oggi 21:2348, 2003.

### Conopeptides Having Novel Amino Acid Sequences

The invention relates to a novel class of conopeptides having several features that set them apart from all other previously described classes of conopeptides. As described in detail in the Examples below, these peptides were isolated and characterized from the venom of cone snails of the species Conus gladiator and Conus mus. Using a combination of protein purification and analytical techniques, a novel class of conopeptides was identified having the following general structure:

| | |
|---|---|
| A-O-X₁-N-S-X₂-W-S wherein: | (SEQ ID NO:1) |

O is γ-hydroxyproline;
X₁ is A or S;
X₂ is V or γ-hydroxyvaline (V*); and
W is D-Tryptophan

This general structure defines a motif that was conserved in native octapeptides isolated from two evolutionarily diverse species that inhabit different geographic regions, i.e., Conus gladiator and Conus mus. The structure of these peptides is distinguished by the presence of unusual amino acid modifications, i.e, hydroxyproline in residue 2 and D-Trp in residue 7. Within this novel class of peptides are found two subclasses of native octapeptides which differ between the two species by only one amino acid, i.e., at residue 2 (X₁). This variation at residue 2 is a conservative change from an Alanine in the gla peptide subclass to a Serine in the mus subclass of peptides. Additionally, peptides which are believed to be the mature, most active forms include hydroxyvaline in residue 6 (X₂), whereas their presumed precursors contain unhydroxylated valine at this position.

Even without their unusual post-translational modifications, the native gla and mus conophan peptides both exhibit primary sequence structures not found in any known or hypothetical protein or peptide, as shown below:

| | |
|---|---|
| gla-1/gla-1′-gla-2: A-P-A-N-S-V-W-S | (SEQ ID NO:2) |
| mus-1/mus-1′/mus-2: A-P-S-N-S-V-W-S | (SEQ ID NO:3) |

Conservation of the highly similar amino acid backbone in the two subclasses of octa-conopeptides suggests that this backbone is likely to form an important scaffold for preservation of the function of these molecules. As used herein, the term “backbone amino acid sequence” refers to the primary structure of the peptide prior to post-translational modification.

### Conopeptides Containing Hydroxylated Amino Acids

Many preferred embodiments of the peptides contain post-translational modifications that were discovered in the native conopeptides. The name given to one of the new classes of compounds, i.e., the γ-Hydroxyconophans, is derived in part from the finding of a post-translation modification of hydroxylation of valine in the gamma position in native gla-1 and mus-1 peptides. (Conophans, exemplified by gla-2 and mus-2, by contrast, lack the hydroxylation on the valine residue, and are thought to be the precursors of gla-1 and mus-1, respectively, which are acted upon by a presumed valine hydroxylase enzyme to form the mature γ-Hydroxyconophans.) In the γ-Hydroxyconophan class of conopeptides, the residue in position 6 is γ-Hydroxyvaline (γ-Hyv; V*).

Beyond the hydroxylation of Pro, we have discovered a new family of conopeptides that have as a feature the hydroxylation of Val. We isolated four novel conopeptides: gla-1/gla-1′ from the venom of Conus gladiator and mus-1/mus-1′ from venom of Conus mus. These conopeptides contain the modified amino acid γ-hydroxyvaline (Hyv=V*). The complete sequences of these conopeptides, gla-V*/gla-V*′=Ala-Hyp-Ala-Asn-Ser-D-Hyv-Trp-Ser and mus-V*/mus-V*′=Ala-Hyp-Ser-Asn-Ser-D-Hyv-Trp-Ser, were determined by a combination of nano-NMR and MS/MS methods. Additionally, we have isolated three related conopeptides from Conus vilepinii (vil-M, vil-I and vil-I(O2P), that are similar in sequence and properties to the gla/mus conophans; however, vil-M and vil-I incorporate D-Ile and D-Met instead of D-Val. The vil-I(O2P) suggests a hierarchical set of posttranslational modifications, where epimerization precedes hydroxylation and hydroxylation of Pro precedes hydroxylation of D-Val. These are the first examples of a polypeptide chains containing Hyv and it suggests the existence of a corresponding enzyme capable of D-Val oxidation. These conopeptides are particularly unusual because (i) they are not constrained as most conopeptides (ii) they are hyperhydroxylated as they have a high content of hydroxylated residues, and (iii) their sequences have no close match with other peptides in any sequence database.

The Ser-D-γ-Hyv-Trp triad is an unusually stable structural motif that incorporates the unprecedented modification of a D-amino acid and has produced the first examples of hydroxyvaline within polypeptide chains. This motif defines the new class of conopeptides: γ-hydroxyconophans. The corresponding precursors, such as gld-V, are termed conophans. The presence of γ-hydroxyvaline in gld-V* as opposed to valine in gld-V suggests the existence of a corresponding enzyme capable of D-Val oxidation. This putative enzyme could be using gla-V, or its precursor protein, as a substrate to modify the specified D-Val to generate the final form of the toxin. This process challenges previous understanding of homochirality in living organisms, as all known enzymatic reactions acting on peptides and proteins are stereoselective for the L-amino acids within them.

Based on studies described in Examples below, the stability of γ-Hyv within these conopeptides is thought to be preserved by the presence of D-Trp as the adjacent residue. This dyad of modified amino acids represents a novel structural motif that characterizes the γ-Hydroxyconophan class of conopeptides. Accordingly, the γ-Hydroxyconophans can be described by the general structural formula:

| | |
|---|---|
| A-O-X₁-N-S-V*-W-S | (SEQ ID NO:4) |

wherein:
O=γ-hydroxyproline;
V*=γ-hydroxyvaline; and
W=D-Trp.

In preferred embodiments, the conopeptides are identified by any one of SEQ ID NO's.: 1-54.

In another preferred embodiment, the conopeptides comprise Ser-D-γ-OH-Xaa-Trp, wherein Xaa=Asp, Asn, Ser, Thr, Val, Ala, Gly, Leu, Ile, or Pro in the D- or L-configuration.

Naturally occurring members of this class include the following octapeptides:

| | | |
|---|---|---|
| gla-1/gla-1′: | A-O-A-N-S-V*-W-S | (SEQ ID NO:5) |
| mus-1/mus-1′ | A-O-S-N-S-V*-W-S | (SEQ ID NO:6) |

wherein:
O=γ-hydroxyproline;
V*=γ-hydroxyvaline; and
W=D-Trp.

Prior to the demonstration herein, γ-Hyv had not been found within a polypeptide chain (Hernandez, I. L. C., Godinho, M. J. L., Magalhaes, A., Schefer, A. B., Ferreira, A. G., Berlinck, R. G. S. Journal of Natural Products 2000, 63, 664-665; Krishna, R. G., Wold, F. In Proteins: Design and Analysis; Angeletti, R. H., Ed.; Academic Press: San Diego, Calif., 1998, pp 121-206). This is not surprising for the following reason. The hydroxylation of valine would be an unexpected post-translational modification in proteins and peptides because the hydroxyl group could readily cleave the peptide bond by intraresidue cyclization to form a lactone. Applicants, not seeking to be bound to this or any other theory, present a mechanism for the existence of γ-Hyv in the γ-Hydroxyconophan octopeptide relating to its proximity to the contiguous D-Trp in residue 7.

The presence of hydroxlated amino acids (i.e., in position 2 and position 6) is a prominent feature of the γ-Hydroxyconophans and is believed to impart structural stability to these molecules. Additionally, it is generally agreed that the hydroxylation which is observed in many classes of known conopeptides may be related to hydrogen bonding directed towards increasing binding strength and selectivity towards their neuronal targets, which are typically cell surface receptors. Although preferred embodiments of the peptides are described as having hydroxylated proline and valine, hydroxylated amino acids in general are known as an important class of modified amino acids in proteins. For example, γ-Hydroxyproline (γ-Hyp) and δ-hydroxylysine (δ-Hyl) are commonly found in collagen and are vital for collagen structural stability (Haading, J. J., Crabbe, M. J. C. Post-translational Modifications of Proteins, CRC Press: Boca Raton, Fla., 1992; Perret, S., Merle, C., Bemocco, S., Berland, P., Garrone, R., Hulmes, D. J. S., Theisen, M., Ruggiero, F. J. Biol. Chem. 2001, 276, 43693-43698). γ-Hydroxyarginine has been found as part of the sequence of polyphenolic proteins that form the adhesive plaques of marine mussel species (Papov, V. V., Diamond, T. V., Biemann, K, Waite, J. H. J. Biol. Chem. 1995, 270, 20183-20192). Accordingly, those of skill in the art will appreciate that peptides based on the disclosed amino acid backbones and post-translation modifications could readily be envisoned and produced by substitution with other hydroxylated amino acids without departing from the spirit and scope of the invention.

### Synthesis of Conophan and γ-Hydroxyconophan Conopeptides

As shown in Examples below, the peptides of the invention can be synthesized starting from amino acids by methods well known in the art. Alternatively, it will be readily apparent to those of skill in the art of molecular biology that cDNA molecules can be isolated that encode the venom proteins (that are subsequently cleaved to form the active conopeptides), starting with PCR primers based on the disclosed amino acid sequences of the peptides. Using well known methods, the cDNAs can be used, for example, to transfect cells for in vitro or in vivo production of recombinant proteins and peptides.

The peptides of the invention can vary in length, but are preferably between about 5 and 200 amino acids in length, more preferably between about 5 and 50 amino acids in length, and most preferably between about 5 and 25 amino acids in length. The peptides can be modified using many methods standard in the art depending upon their intended use.

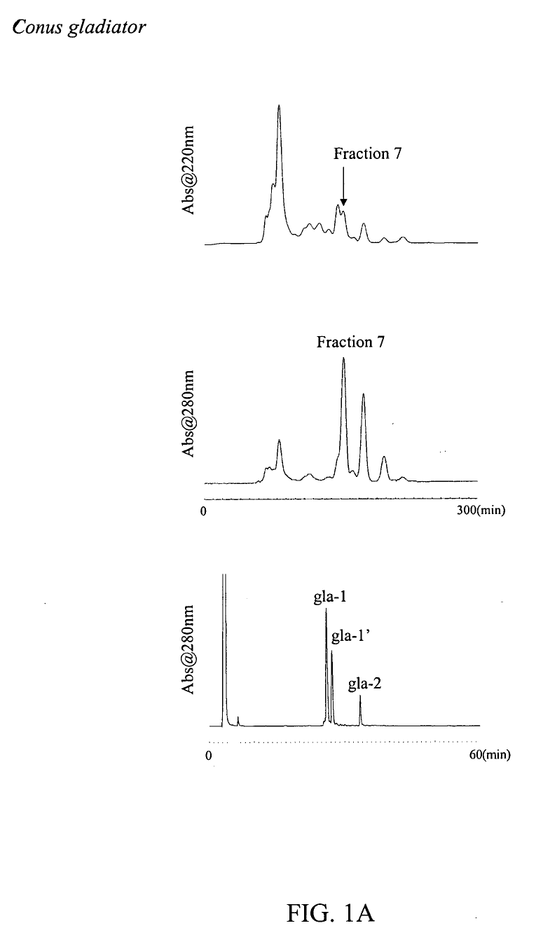

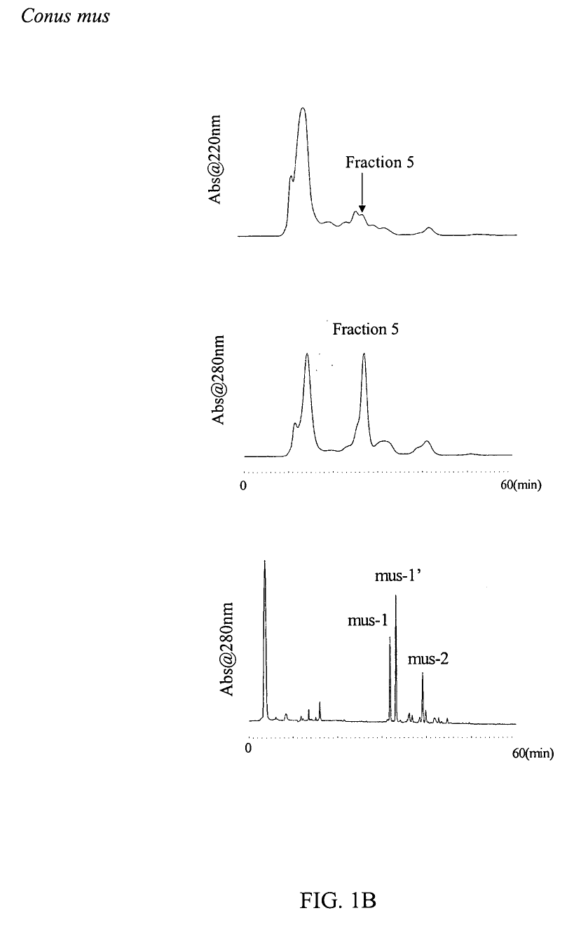

As described in detail in the Examples which follow, the analysis and characterization of conopeptides isolated from cone snail species from the Americas, Conus regius (species code: reg), a widespread worm-hunting cone snail of the Western Atlantic Ocean (FIG. 1). The results from the isolation and structural analysis of three hydroxylated conopeptide families from Conus regius: α-conotoxins, the newly described mini-M conotoxins and a novel family of linear conopeptides that we have termed 6γ, are described in the Examples which follow. These peptides exhibit a differential proline hydroxylation strategy that is likely to affect their neuronal targeting. Beyond the hydroxylation of Pro, we have discovered a new family of conopeptides that feature the modified amino acid D-γ-hydroxyvaline (Hyv=V*). This doubly modified amino acid is part a novel structural motif, Ser-D-γ-OH-Xaa-Trp, that defines a new class of conopeptides that we have termed γ-hydroxyconophans. Hydroxyconophans constitute the first examples of a polypeptide chains containing Hyv. We have also isolated analogous conopeptides containing D-Val instead of D-Hyv; these are termed conophans. Additionally, we have isolated three related conopeptides from Conus vilepinii (vil-M, vil-I and vil-I(O2P) that are similar in sequence and properties to the gla/mus conophans; however, vil-M and vil-I incorporate D-Ile and D-Met instead of D-Val.

### Utility of γ-Hydroxyconophans and Conophans

As members of a new class of conopeptides, the γ-Hydroxyconophans and Conophans can be used as agents that selectively bind to and thereby modulate the function of specific cell surface receptors on cells, for example on neurons. Various classes of conopeptides are known to selectively bind to cellular receptors including numerous subclasses of acetylcholine receptors, subtypes of ion channels including sodium, potassium, calcium and chloride channels, glutamate receptors, N-Methyl-D-Aspartate (NMDA) receptors and neurotensin receptors. As shown in Examples below, gla-1 and gla-2 peptides derived from the octapeptide sequences of gla-1 and gla-2 were shown to modulate Ca⁺⁺ flux in cultured neurons from rat cortex. This effect was presumed to be mediated through cell surface receptors that either directly or indirectly control calcium ion concentration in the cells.

Accordingly, in one aspect the invention provides a method for modulating the level of an ion within a cell. The method includes the steps of: (a) providing a cell that responds to a peptide that binds to a chemical structure on the surface of the cell by modulating the level at least one ion within the cell; and (b) contacting the chemical structure on the surface of said cell with a peptide containing the amino acids γ-OH-Val and D-Trp in contiguous residues, wherein the peptide selectively binds to the chemical structure. An exemplary chemical structure on the cell surface is a receptor, of which many different types are known, as described above, including inter alia calcium channels, sodium channels, potassium channels and chloride channels. Selective binding of particular classes of receptors by the peptides of the invention can be determined empirically by testing the peptides in receptor assay systems that are well established for this purpose.

### Animal Subjects

Because control of ion flux in cells is central to a very large number of physiological processes in animals and man, the invention is believed to be compatible with any animal subject. A non-exhaustive list of examples of such animals includes mammals such as mice, rats, rabbits, goats, sheep, pigs, horses, cattle, dogs, cats, and primates such as monkeys, apes, and human beings. Those animal subjects that have a disease or condition that relates to modulation of calcium levels within a cell are preferred for use in the invention, as these animals may have the symptoms of their disease reduced or even reversed. In particular, human patients suffering from neurologic disorders such as Alzheimer's disease, immune system dysfunction, and cancer might particularly benefit.

### Administration of Compositions

The compositions of the invention may be administered to animals including humans in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. Such administration may be oral or parenteral (for example, by intravenous, subcutaneous, intramuscular, or intraperitoneal introduction). The compositions may also be administered directly to the target site by, for example, surgical delivery to an internal or external target site, or by catheter to a site accessible by a blood vessel. Other methods of delivery, for example, liposomal delivery or diffusion from a device impregnated with the composition, are known in the art. The compositions may be administered in a single bolus, multiple injections, or by continuous infusion (for example, intravenously or by peritoneal dialysis). For parenteral administration, the compositions are preferably formulated in a sterilized pyrogen-free form.

Compositions of the invention can also be administered in vitro to a cell (for example, in in vitro assays to modulate calcium levels within the cells, or to target particular cell surface receptors capable of selectively binding these peptides).

The compositions comprising any one of SEQ ID NO's.: 1-54, as the active ingredient, and pharmaceutical acceptable salts. Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion. For example, administration of the active agent according to this invention may be achieved using any suitable delivery means, including:
(a) pump (see, e.g., Annals of Pharmacotherapy, 27:912 (1993); Cancer, 41:1270 (1993); Cancer Research, 44:1698 (1984));
(b) microencapsulation (see, e.g., U.S. Pat. Nos. 4,352,883; 4,353,888; and 5,084,350);
(c) continuous release polymer implants (see, e.g., U.S. Pat. No. 4,883,666);
(d) macroencapsulation (see, e.g., U.S. Pat. Nos. 5,284,761, 5,158,881, 4,976,859 and 4,968,733 and published PCT patent applications WO92/19195, WO 95/05452);
(e) naked or unencapsulated cell grafts to the CNS (see, e.g., U.S. Pat. Nos. 5,082,670 and 5,618,531);
(f) injection, either subcutaneously, intravenously, intra-arterially, intramuscularly, or to other suitable site; or
(g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines, other conopeptides and other therapeutic agents useful in all of the major areas of clinical medicine. When used with other pharmaceutically active compounds, the conopeptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

As disclosed herein, the compounds and compositions of the present invention are useful in treating pain. As such, they may also be useful in treating inflammatory pain. Accordingly, the compounds and compositions of the present invention may also be utilized to treat numerous inflammatory disease states and disorders other than pain. For example, the compositions and compounds may be useful for treating disorders or diseases including but not limited to: Alzheimer's disease, multiple sclerosis, attenuation of morphine withdrawal, cardiovascular changes, oedema, such as oedema caused by thermal injury, chronic inflammatory diseases such as rheumatoid arthritis, asthma/bronchial hyperreactivity and other respiratory diseases including allergic rhinitis, inflammatory diseases of the gut including ulcerative colitis and Crohn's disease, ocular injury and ocular inflammatory diseases, proliferative vitreoretinopathy, irritable bowel syndrome and disorders of bladder function including cystitis and bladder detrusor hyperreflexia, demyelinating diseases such as multiple sclerosis and amyotrophic lateral sclerosis, asthmatic disease, small cell carcinomas, in particular small cell lung cancer, depression, dysthymic disorders, chronic obstructive airways disease, hypersensitivity disorders such as poison ivy, vasospastic diseases such as angina and Reynauld's disease, fibrosing and collagen diseases such as scleroderma and eosinophilic fascioliasis, reflex sympathetic dystrophy such as shoulder/hand syndrome, addiction disorders such as alcoholism, stress related somatic disorders, neuropathy, neuralgia, disorder related to immune enhancement or suppression such as systemic lupus erythmatosis conjunctivitis, vernal conjunctivitis, contact dermatitis, atopic dermatitis, urticaria, and other eczematoid dermatitis and emesis; central nervous system disorders such as anxiety, depression, psychosis and schizophrenia; neurodegenerative disorders such as AIDS related dementia, senile dementia of the Alzheimer type, Alzheimer's disease and Down's syndrome; demyelinating diseases such as multiple sclerosis (MS) and amyotrophic lateral sclerosis (ALS; Lou Gehrig's disease) and other neuropathological disorders such as peripheal neuropathy, inflammatory diseases such as inflammatory bowel disease, irritable bowel syndrome, psoriasis, fibrositis, ocular inflammation, osteoarthritis and rheumatoid arthritis; allergies such as eczema and rhinitis; hypersensitivity disorders such as poison ivy; ophthalmic diseases such as conjunctivitis, vernal conjunctivitis, dry eye syndrome, and the like; cutaneous diseases such as contact dermatitis, atopic dermatitis, urticaria, and other eczematoid dermatitis; oedema, such as oedema caused by thermal injury; addiction disorders such as alcoholism; stress related somatic disorders; reflex sympathetic dystrophy such as shoulder/hand syndrome; dysthymic disorders; neuropathy, such as diabetic or peripheral neuropathy and chemotherapy-induced nemopathy; postherpetic and other neuralgias; asthma; osteoarthritis; rheumatoid arthritis; migraine reperfusion injury to an ischemic organ, e.g., reperfusion injury to the ischemic myocardium, myocardial infarction, inflammatory bowel disease, rheumatoid arthritis, osteoarthritis, hypertension, psoriasis, organ transplant rejections, organ preservation, impotence, radiation-induced injury, asthma, atherosclerosis, thrombosis, platelet aggregation, metastasis, influenza, stroke, burns, trauma, acute pancreatitis, pyelonephritis, hepatitis, autoimmune diseases, insulin-dependent diabetes mellitus, disseminated intravascular coagulation, fatty embolism, adult and infantile respiratory diseases, carcinogenesis and hemorrhages among many others.

### Effective Doses

An effective amount is an amount which is capable of producing a desirable result in a treated animal or cell (for example, reduced calcium flux in the cells of the animal or in a cell in culture). As is well known in the medical and veterinary arts, dosage for any one animal depends on many factors, including the particular animal's size, body surface area, age, the particular composition to be administered, time and route of administration, general health, and other drugs being administered concurrently. It is expected that an appropriate dosage for parenteral or oral administration of compositions of the invention would be in the range of about 1 μg to 100 mg/kg of body weight in humans. An effective amount for use with a cell in culture will also vary, but can be readily determined empirically (for example, by adding varying concentrations to the cell and selecting the concentration that best produces the desired result). It is expected that an appropriate concentration would be in the range of about 0.0001-100 mM. More specific dosages can be determined, for example, using a cultured neuronal cell assay as described below.

The active agent is preferably administered in an therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Dosage may be adjusted appropriately to achieve desired drug levels, locally or systemically. Typically the conopeptides of the present invention exhibit their effect at a dosage range from about 0.001 mg/kg to about 250 mg/kg, preferably from about 0.05 mg/kg to about 100 mg/kg of the active ingredient, more preferably from a bout 0.1 mg/kg to about 75 mg/kg, and most preferably from about 1.0 mg/kg to about 50 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example 24 hours or multiple doses per day are contemplated to achieve appropriate systemic levels of compounds.

Toxicity and efficacy of the compositions of the invention can be determined by standard pharmaceutical procedures, using cells in culture and/or experimental animals to determine the LD₅₀ (the dose lethal to 50% of the population) and the ED₅₀ (the dose that effects the desired result in 50% of the population). Compositions that exhibit a large LD₅₀/ED₅₀ ratio are preferred. Although less toxic compositions are generally preferred, more toxic compositions may sometimes be used in in vivo applications if appropriate steps are taken to minimize the toxic side effects.

Data obtained from cell culture and animal studies can be used in estimating an appropriate dose range for use in humans. A preferred dosage range is one that results in circulating concentrations of the composition that cause little or no toxicity. The dosage may vary within this range depending on the form of the composition employed and the method of administration.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The present invention is further illustrated by the following specific examples, which should not be construed as limiting the scope or content of the invention in any way.

Materials and Methods

Specimen Collection

Specimens of C. villepinii were collected in the Florida Keys, USA, dredging at depths ranging from 100-200 m. Additional snails were collected using a deep submersible vehicle at 200 m at the same location indicated above. Snails were collected using a suction device attached to a robotic arm. The snails were immediately frozen and stored at −80° C. Specimens of C. floridanus floridensis were collected in the southwest coast of Florida, USA, in sandy areas at low tides and immediately frozen and stored at −80° C.

Crude Venom Extraction

Venom ducts dissected from either 63 specimens of C. villepinii or 47 specimens of C. floridanus floridensis were homogenized in 0.1% trifluoroacetic acid (TFA) at 4° C. Whole extracts were centrifuged at 10,000×g for 20 min, at 4° C., and the resulting pellets were washed three times with 0.1% TFA and re-centrifuged under identical conditions. The supernatants containing the soluble peptides were pooled, lyophilized, and stored at −80° C. until further use.

Peptide Purification

Crude venoms were fractionated by high-performance liquid chromatography (HPLC) on a size exclusion column (Pharmacia Superdex-30, 2.5×100 cm), which was equilibrated and eluted with 0.1M NH₄HCO₃ using a flow rate of 1.5 ml/min. Chromatographic fractions were monitored at 220, 250 and 280 nm. Additional purification of peptide-containing peaks was achieved by reverse-phase HPLC on a C18 semipreparative column (Vydac, 218TP510, 10×250 mm; 5 μm particle diameter; 300 Å pore size) equipped with a C18 guard column (Upchurch Scientific, AC-43 4.6 mm) at a flow rate of 3.5 ml/min. Further purification of the peptides was achieved by re-chromatography employing an analytical C18 column (Vydac, 238TP54, 4.6×250 mm; 5 μm particle diameter; 300 Å pore size), with a flow rate of 1 ml/min. For both semipreparative and analytical RP-HPLC, the buffers were 0.1% TFA (buffer A) and 0.1% TFA in 60% acetonitrile (buffer B). The peptides were eluted with a linear gradient of 1% buffer B increase/min. Absorbance were monitored at 220 and 280 nm. Peptide peaks were manually collected, lyophilized and kept at −40° C.

Reduction and Alkylation of Cysteyl Residues

Reduction and alkylation of peptide cysteines was carried out as previously described (Yen et al., 2002, Journal of Mass Spectrometry 37, 15-30) with slight modifications. An aliquot of each peptide (˜1 pmol) was dried, re-dissolved in 0.1 M Tris-HCl (pH 6.2), 5 mM EDTA, 0.1% sodium azide and reduced with 6 mM dithiothreitol (DTT). Following incubation at 60° C., for 30 min, peptides were alkylated at a final volume of 15111 with 20 mM Iodoacetamide (IAM) and 2 μl of NH₄OH (pH 10.5), at room temperature, for 1 hour, in the dark. The reduced and alkylated peptides were purified using a Zip Tip (C18, size P10, Millipore).

Peptides Sequencing

Alkylated peptides were adsorbed onto Biobrene-treated glass fiber filters and amino acid sequences were acquired by Edman degradation using an Applied Biosystems Procise model 491A Sequencer.

Molecular Mass Determination

Positive ion matrix laser desorption ionization-time of fly (MALDI-TOF) mass spectrometry were measured with a Voyager-DE STR from Applied Biosystems. Samples were dissolved in 0.1% TFA, 50% acetonitrile, and applied on α-cyano-4-hydroxycinnamic acid matrix. Spectra were obtained in the linear and reflector mode using Calmix 1 and Calmix 2 (Applied Biosystems) as external calibration standards.

Disulfide Connectivity Analysis

About 1 nmol of lyophilized peptide was digested with a combination of cyanogen bromide (CNBr) and α-Chymotrypsin prepared free of autolysis products and low molecular weight contaminants. In this case, peptides were not previously reduced and alkylated in order to maintain intact their disulfide bonds. Lysis of the peptides with CNBr (Acros Organics, USA) was performed using a ratio of 2 mg of CNBr per milligram of peptide (Simpson, 2003), in 20 μl of 70% formic acid. The peptidic sample was incubated for 20 h, at room temperature, in the dark, and the reaction was terminated by diluting the mixture with ˜5 volumes of H₂O following by removal of the excess free reagents by lyophilization with a Speed Vac concentrator. Subsequently, the sample was further digested with chymotrypsin (Sigma-Aldrich, USA), using a 1:20 ratio of chymotrypsin to substrate, in 20 μl of 0.1M NH₄HCO₃ (pH 8.0), for 4 h, at 37° C. Final digests were directly analyzed by MALDI-TOF mass spectrometry.

Circular Dichroism Spectroscopy

All circular dichroism (CD) measurements were carried out with a JASCO J-810 spectropolarimeter instrument using a 0.2 ml solution of each peptide in high purity water (concentration 3 μM) in a quartz cell. Spectra were recorded over a 190-250 nm range at 25° C. as an average of 10 scans at a scan speed of 100 nm/min.

NMR Spectroscopy

Peptides were analyzed by 1D and 2D NMR spectroscopy using a Varian Inova 500 MHz instrument equipped with pulse field gradients, three radiofrequency channels and waveform generators. Native peptides were dissolved in high purity water containing 10% D₂O, and 38 pmol of trimethyl silyl propionic acid (TSP) as an internal reference. The final sample solution volume was 40 μl at a pH of 3.6. Spectra were obtained using a 1.7 mm NMR capillary tube (Wilmad), at 0 and 25° C. Water suppression was carried out using Watergate (wg) for pulse sequences 2D experiments TOCSY and, NOESY.

Nomenclature

To distinguish clones from peptides that have been isolated from Conus crude venom, the species letter(s) are lower case, Arabic number represents the disulfide framework and a letter indicates the order of discovery of each peptide. If a target is assigned to a toxin, the appropriate pre-existing or newly Greek letter will prefix the name (Craig et al., 1999, J. Biol. Chem. 274, 30664-30671). As used herein, a nomenclature of three letters was adopted because the two letters nomenclature would not be enough to describe the large quantities of different non-fish-hunting species, especially those with similar first letters name. The three letters “flf” were used to name the peptides for C. floridanus floridensis and “vil” to C. villepinii. A number “13” represents the disulfide framework based on the last number for conotoxin that was XII (Olivera B., 2002, Annual Review of Ecology and Systematics, 33, 25-47). Roman numbers were not adopted because these peptides were isolated from the crude venom and not from the DNA.

### EXAMPLE 1

### Collection of Conus Specimens and Venom

Specimen collection and venom isolation. Conus gladiator is a predatory worm-hunting cone species that inhabits shallow water along the Pacific coast from the Gulf of California to Peru. Specimens were collected from several locations off the Pacific coast of Costa Rica. The snails were dissected to obtain their venom ducts. Crude venom extraction was performed in H₂O/Acetonitrile (40/60)+0.1% TFA. The venom extract was filtered and centrifuged at 10,000 rpm at 5° C. for 10 min. The supernatant was pooled and lyophilized (˜50 mg from 47 snails) and used for subsequent analyses. Specimens of Conus mus were collected off the Florida Keys (Plantation, Monroe County). Crude venom (12 mg) was extracted from these snails and processed as described for Conus gladiator.

### EXAMPLE 2

### Chromatographic Separation of Conus Venom

Peptide isolation. Initial fractionation of the venom lyophylates was performed using SE-HPLC on a Pharmacia Superdex-30 column (2.5×100 cm) with elution by 0.1 M NH₄HCO₃ buffer at a flow rate of 1.5 ml/min. The column eluent was monitored on a PDA detector (TSP SM-5100) at λ=220 and 280 nm. The Trp-containing fractions in the major peak (gla_{—}09) in the λ=280-detected chromatogram were further separated using an RP-HPLC Vydac C18 column (10×250 mm, 5 μm, 300 Å) eluted with a linear gradient of H₂O/60% CH₃CN over 100 min. TFA (0.1%) was used as ion-pairing reagent.

### EXAMPLE 3

### 1D and 2D NMR Spectroscopy of Conus Peptides

NMR spectra were acquired on a Varian Inova 500 MHz spectrometer (Varian, Palo Alto, Calif.) equipped with three RF channels, pulse field gradients and waveform generators. Initially, 1D and 2D-TOCSY spectra were recorded using a gHX HR-MAS probe for 1 nmol of gla-1 in 35 μl. The gla conopeptides were isolated in nanomolar quantities, whereas the mus conopeptides were isolated in picomolar quantities. The use of a High Resolution Magic Angle Spinning (HR-MAS) probe (Barbara, T. M. Journal of Magnetic Resonance Series A 1994, 109, 265-269; Barbara, T. M., Bronnimann, C. E. Journal of Magnetic Resonance 1999, 140, 285-288) along with related NMR techniques (Matei, E., Pflueger, F. C., Franco, A., Cano, H., Mora, D., Mari, F. Paper presented at the 18th APS, 2003) allowed the acquisition of NMR spectra from such limited amounts of material. The larger sample quantities (20-35 nmol of the gla peptides) were analyzed using 3 mm sample tubes in 130 μl of NMR solution in a 5 mm gHCX triple resonance probe. 1D spectra were acquired using 512 scans, whereas 2D spectra were acquired using 96 increments in t1 with 256 scans per increment in a phase sensitive mode. 2D spectra were processed using linear predictions in t1 to 1024 points and transformed to final size of 2k×2k. The 1D spectra of picomolar amounts of the mus conopeptides were acquired overnight using 3 mm sample tubes in 130 μl of NMR solution in a 5 mm gHCX triple resonance probe. All spectra were recorded at 25° C. and 0° C. in an NMR solution that consisted of 90% H₂O/10% D₂O using TSP as an internal standard. The pH of this solution was adjusted to 3.6 using 0.01 M solutions of HCl and NaOH and a Phoenix micro-pH probe. Water suppression was achieved using WATERGATE (Piotto, M., Saudek, V., Sklenar, V. Journal of Biomolecular Nmr 1992, 2, 661-665) and Excitation Sculpting (Callihan, D., West, J., Kumar, S., Schweitzer, B. I., Logan, T. M. Journal of Magnetic Resonance Series B 1996, 112, 82-85) for the 2D experiments and WET (Smallcombe, S. H., Patt, S. L., Keifer, P. A. Journal of Magnetic Resonance Series A 1995, 117, 295-303) and presaturation for the 1D ¹H spectra. The resonance assignments were carried out using standard biomolecular NMR procedures.

### EXAMPLE 4

### Analysis of Conus Peptides by Positive Ion Electrospray Ionization Mass Spectrometry (ESI-MS/MS)

Positive ion electrospray ionization mass spectrometry (ESI-MS/MS) spectra of all conopeptides (gla-1 and gla-2) were obtained on either a Micromass Q-TOF micro (Waters Corporation, Milford, Mass.) or a Finningan LCQ-Deca instrument (Finnigan, Germany). Samples analysed (˜1 pmol) using the Q-TOF instrument were desalted using a C18 ZipTip and analyzed using a nanospray ion source (Wilm, M., Shevchenko, A., Houthaeve, T., Breit, S., Schweigerer, L., Fotsis, T., Mann, M. Nature 1996, 379, 466469; Wilm, M., Mann, M. Analytical Chemistry 1996, 68, 1-8). Glu-fibrinogen, m/z=785.85 doubly charged, was used as an internal standard. Samples containing ˜10 pmol of peptide were used for analyses using the LCQ instrument. Samples were analyzed by flow injection using 30% ACN/0.1% acetic acid as a carrier. Charged states of the peptides were confirmed by the use of the Zoom-Scan method (van Eijk, H. M. H., Rooyakkers, D. R., Soeters, P. B., Deutz, N. E. P. Analytical Biochemistry 1999, 271, 8-17).

### EXAMPLE 5

### Peptide Synthesis and Sequencing of Conus Peptides by Edman Degradation

Amino acid sequencing was carried out by Edman degradation chemistry on an ABI Procise cLC instrument. Peptide synthesis was performed on an Applied Biosystems 433A peptide synthesizer (Applied Biosystems, Foster City, Calif.). Peptide-resin cleavage utilized appropriate scavengers (King, D. S., Fields, C. G., Fields, G. B. International Journal of Peptide and Protein Research 1990, 36, 255-266; Fields, C. G.; Fields, G. B. Tetrahedron Letters 1993, 34, 6661-6664) to avoid Trp oxidation. The cleaved peptides were purified by HPLC as described above.

### EXAMPLE 6

### Isolation and Chromatographic Separation of gla and mus Peptides from Conus Venom

FIG. 1A shows results of fractionation and purification of the venom of Conus gladiator. Further separation of the material in the major peak (gla-09) in the λ=280-detected chromatogram by RP-HPLC yielded three peptide fractions, i.e., gla-1, gla-1′, and gla-2 (FIG. 1A, lower). Similar profiles were obtained by fractionation of the venom of Conus mus (FIG. 1B).

### EXAMPLE 7

### 1D and 2D NMR Spectroscopy of gla and mus Conopeptides

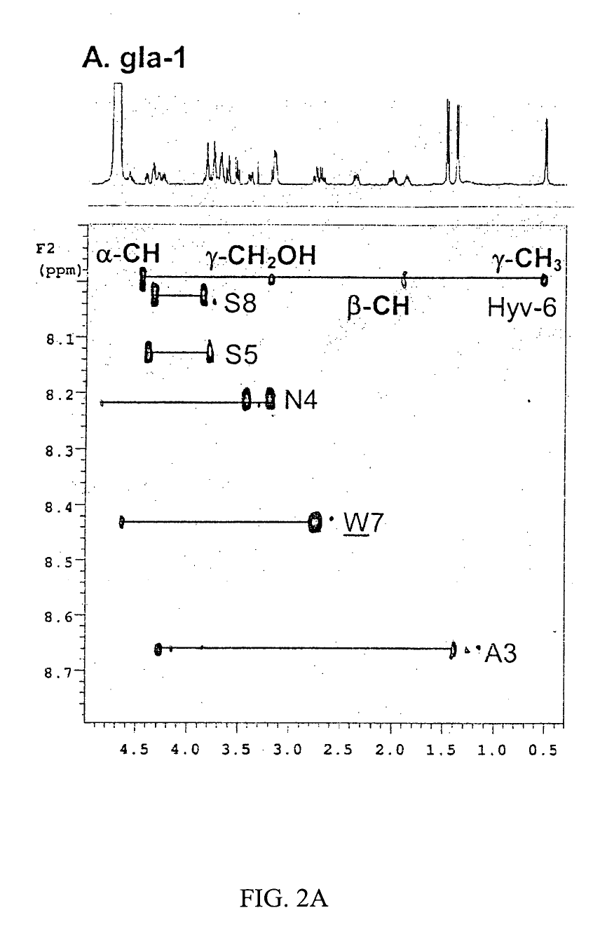

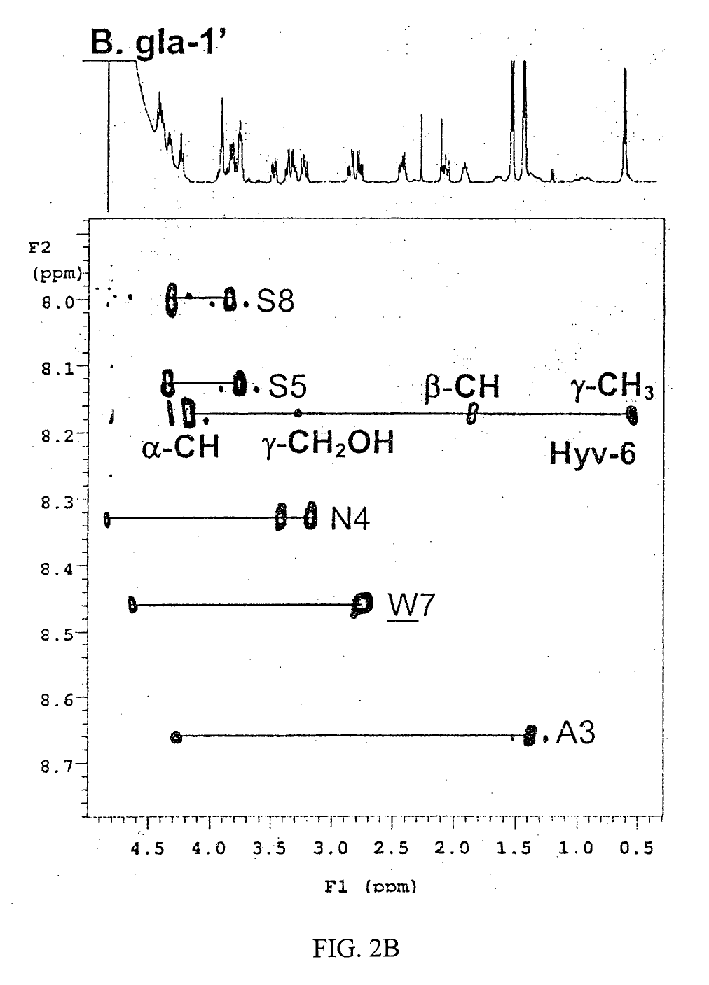

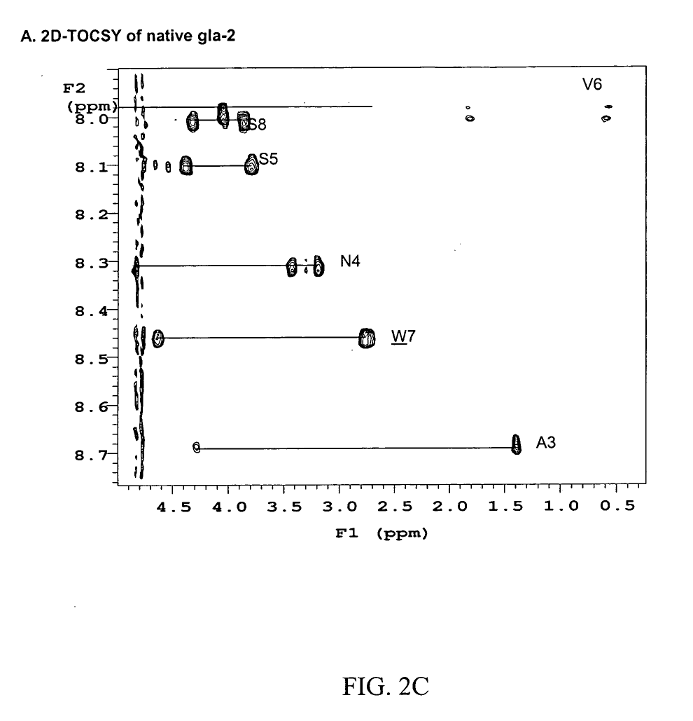

The fractions obtained by RP-HPLC were subsequently analyzed by NMR as described in Example 3 above. Referring to FIGS. 2A-C, the nano-NMR spectra of gla-1 peptides revealed an amino acid composition as follows:
gla-1: 2 Ala, 2 Ser, Trp, Asn, Hyp, and an unusual amino acid (FIG. 2A);
gla-1′: 2 Ala, 2 Ser, Trp, Asn, Hyp, and an unusual amino acid (FIG. 2B); and
gla-2: 2 Ala, 2 Ser, Trp, Asn, Hyp, and Val for gla-2 (FIG. 2C).

### EXAMPLE 8

### MS/MS Spectra of gla and mus Conopeptides

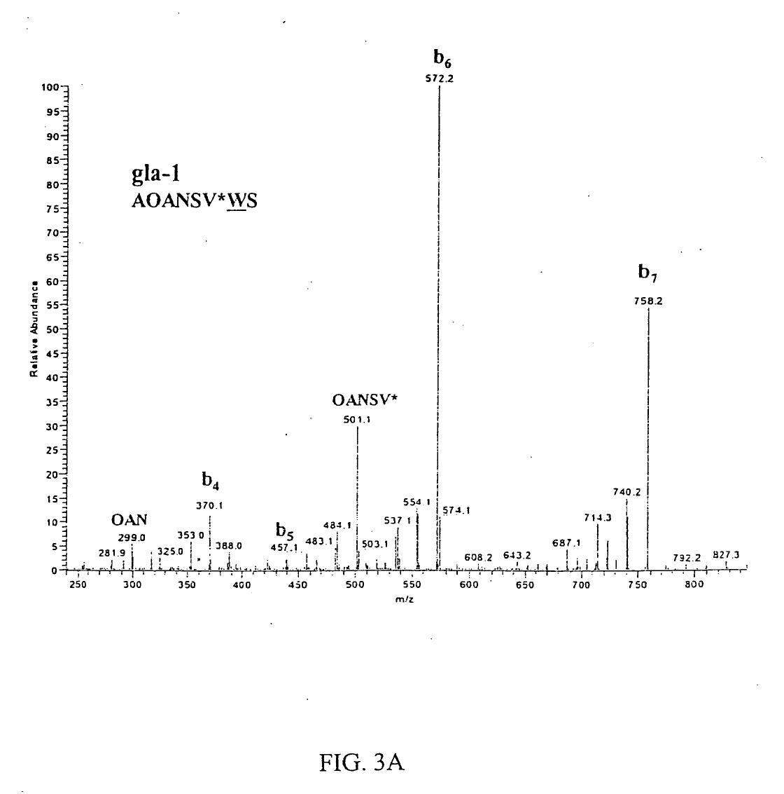

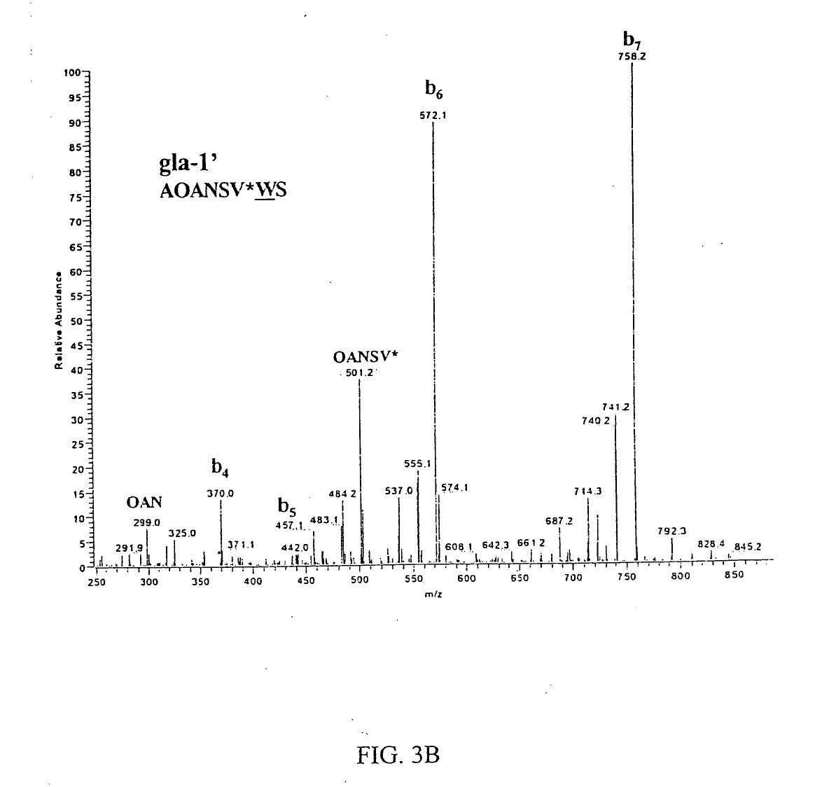

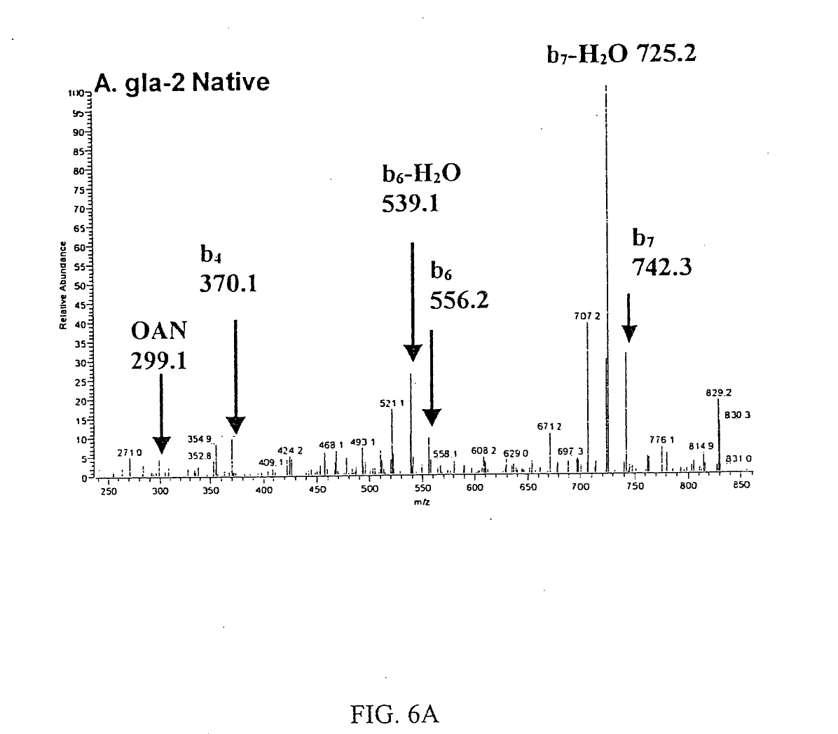

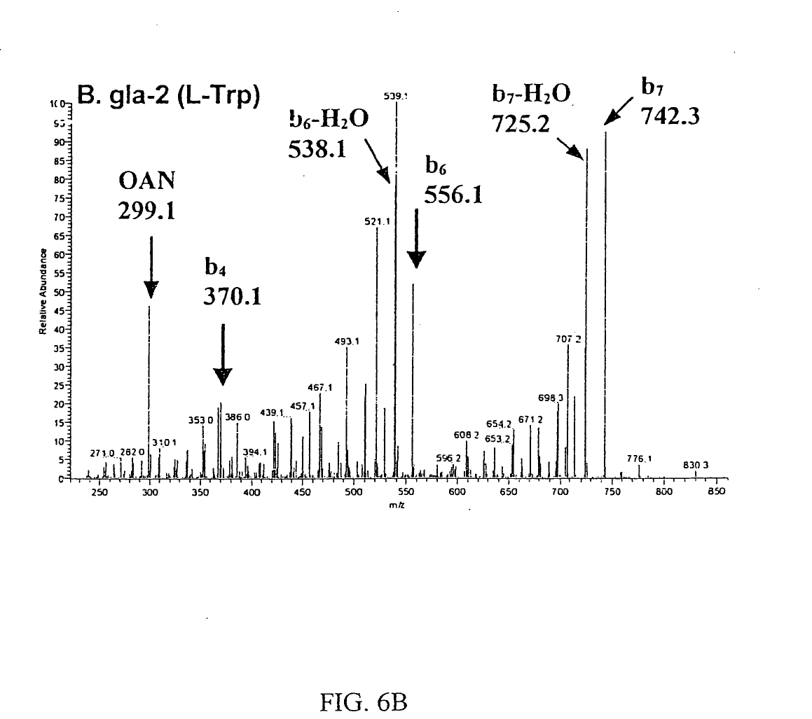

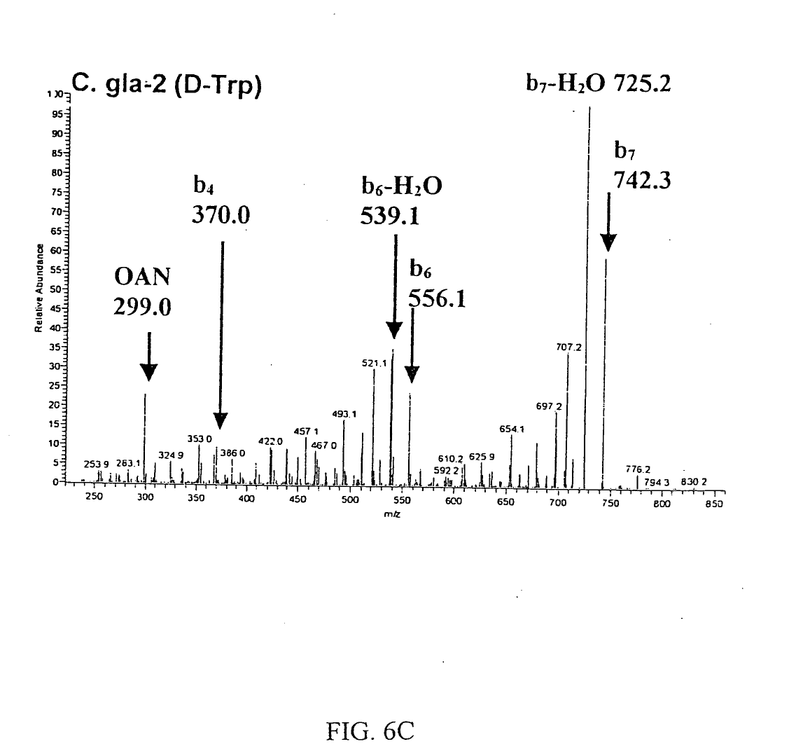

The gla and mus peptides were further analyzed by MS/MS. The mass spectra of gla-1/gla-1′ gave molecular ions of 863.3 Da (FIGS. 3A and 3B). By contrast, the molecular ions of gla-2 were 847.3 Da (FIG. 6A). The NMR and MS data were consistent with gla-1 and gla-1′ having the same covalent structures.

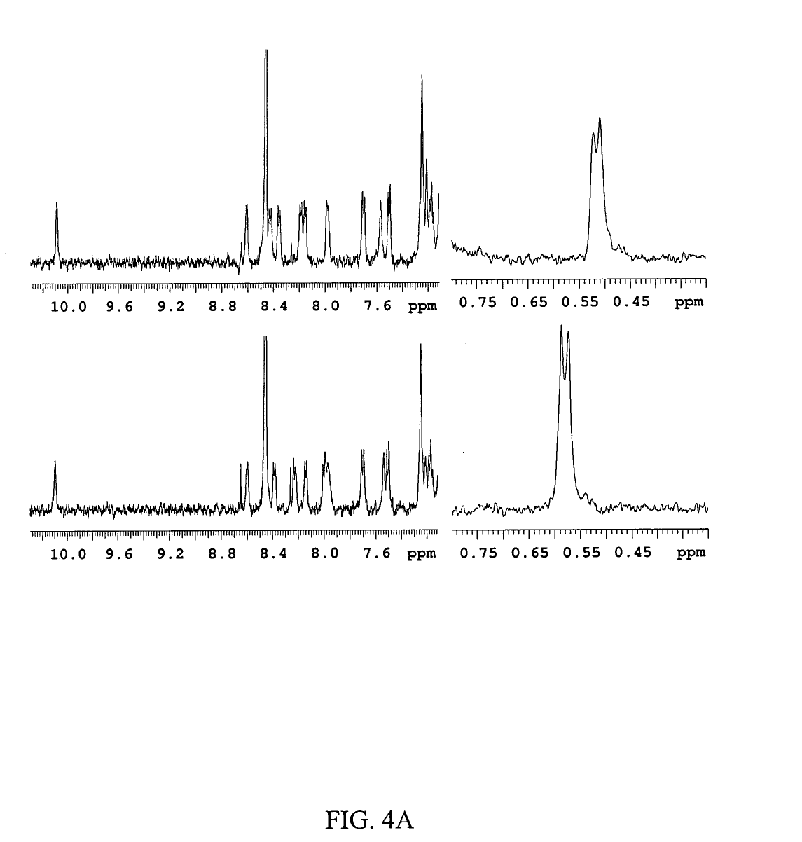

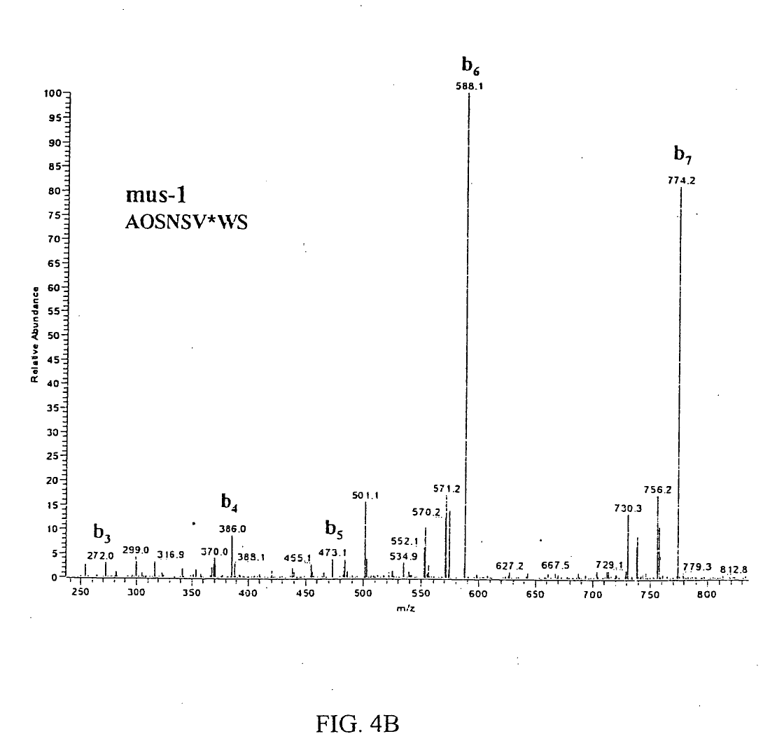

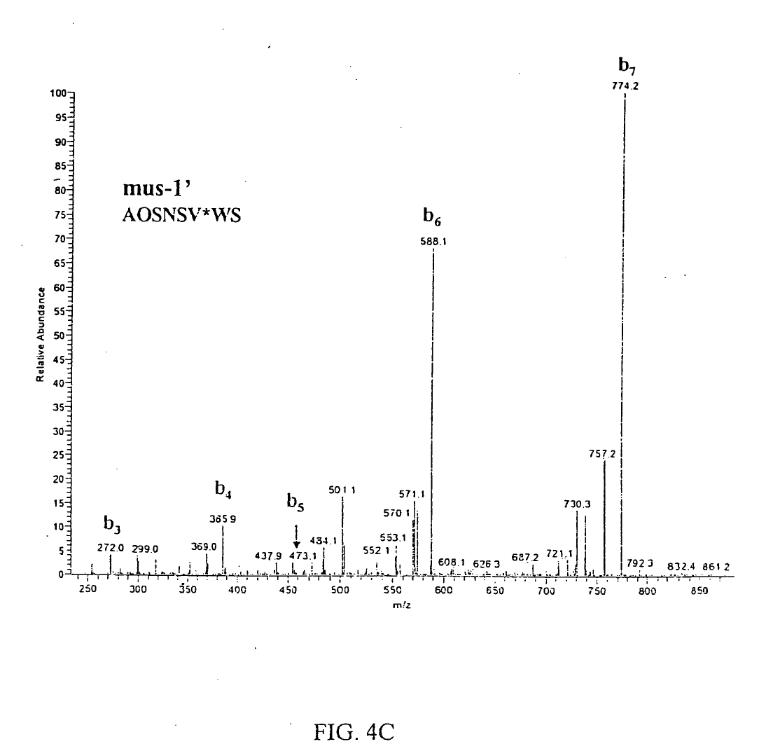

FIGS. 4A-C show corresponding NMR and MS/MS data for the mus octapeptides. The analysis revealed molecular ions of 879.3 Da for the mus-1/mus-1′ pair (FIGS. 4B, 4C) and 863.3 Da for the mus-2 octapeptide.

MS-based analysis revealed the following an amino acid compositions for the mus octapeptides:
mus-1: 3 Ser, Ala, Trp, Asn, Hyp, and an unusual amino acid;
mus-1: 3 Ser, Ala, Trp, Asn, Hyp, and an unusual amino acid; and
mus-2: 3 Ser, Ala, Trp, Asn, Hyp, and Val.

### EXAMPLE 9

### Edman Degradation Analysis of gla and mus Conopeptides

Edman degradation sequence analysis of gla-1 resulted in Ala-Hyp-Ala-Asn-Ser, which is smaller than indicated by the NMR and MS results. By way of contrast, gla-2 was sequenced to completion by Edman degradation chemistry, resulting in a primary structure consistent with the NMR and MS/MS data.

### EXAMPLE 10

### Synthetic Peptides and Confirmation of Presence of D-Trp in γ-Hydroxyconophans

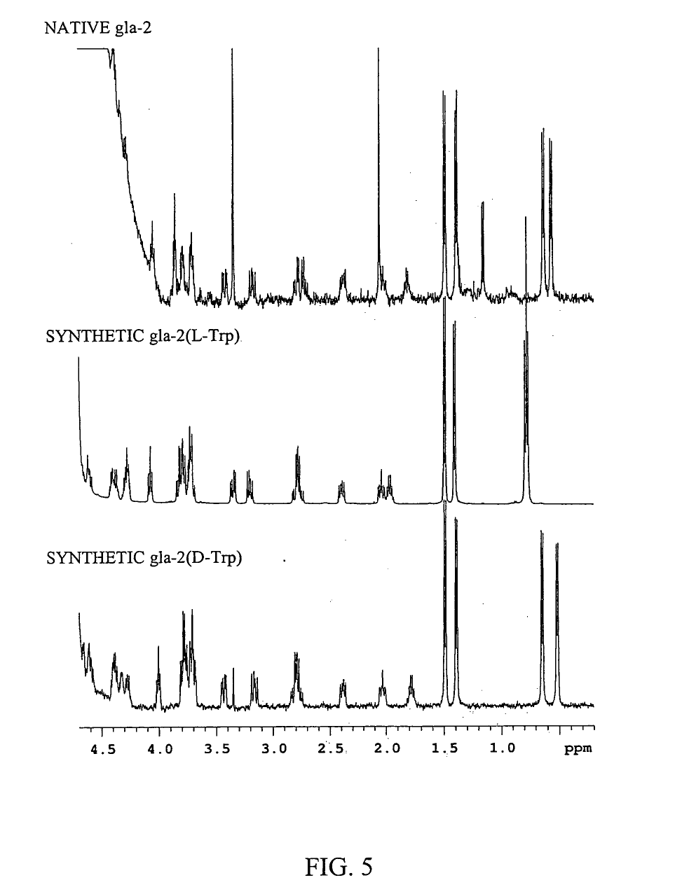

Synthetic γ-Hydroxyconophans corresponding to the gla and mus peptides were synthesized as described in Example 5. To confirm the presence of D-Trp in position 7 of the naturally occurring octapeptides, synthetic gla-2 peptides were constructed having either D-Trp or L-Trp in position 7. FIG. 5 shows 1D NMR chromatographic profiles of native gla-2, and the two synthetic peptides. FIG. 6(A-C) shows a comparison of these peptides by MS/MS. The stereochemistry of the tryptophan residue was determined by comparing the NMR (FIG. 5) and MS/MS spectra of the native gla-2 with its synthetic peptide analogs (FIGS. 6A-C). The strong influence of the chirality of Trp-7 on the MS/MS fragmentation pattern of these peptides was central for the determination.

Results from both of these analyses demonstrated behavior of native gla-2 that was consistent with that of the synthetic sample containing D-Trp. This result thus confirmed the presence of D-Trp in the γ-Hydroxyconophan sequence.

### EXAMPLE 11

### Characterization of Hydroxyvaline (Hyv₆) Diastereomers of gla and mus Conopeptides

As described above, the gla-1/gla-1′ and mus-1/mus-1′ pairs were shown to have the same respective covalent structures. Nevertheless, their chromatographic behaviors revealed differences in hydrophobicity in a temperature-independent fashion. In principle, these differences might be attributed to cis/trans isomerism of the peptide bond involving Hyp in residue 2, as suggested by NMR evidence in other related conopeptides (Pallaghy, P. K., He, W., Jimenez, E. C., Olivera, B. M., Norton, R. S. Biochemistry 2000, 39, 12845-12852). By contrast, ultraviolet resonance Raman spectroscopy suggested that two conformational states within conopeptides could be attributed to the differences of the χ dihedral angles of the D-Trp within their sequence (Jimenez, E. C., Watkins, M., Juszczak, L. J., Cruz, L. J., Olivera, B. M. Toxicon 2001, 39, 803-808). However, in these cases, temperature dependency of the distribution of the conformers has been observed (Jacobsen, R., Jimenez, E. C., Grilley, M., Watkins, M., Hillyard, D, Cruz, L. J., Olivera, B. M. Journal of Peptide Research 1998, 51, 173-179).

To determine the explanation for the differing chromatographic profiles in the case of the gla and mus conopeptide pairs, a detailed analysis of the MS/MS data of the gla and mus γ-Hydroxyconophans was undertaken. The result revealed that the fragmentation patterns within the pairs differs in the intensity of the b₆ fragment (Compare FIGS. 3A, 3B and 6A), suggesting structural differences within the Hyv residue. Furthermore, it was noted that the largest chemical shift differences between gla-1 and gla-1′ are within the resonances of Hyv (FIG. 2). Hyv bears a chiral center at the β carbon; therefore, gla-1 and gla-1′ were postulated to be diastereomers, epimeric at the β carbon of Hyv.

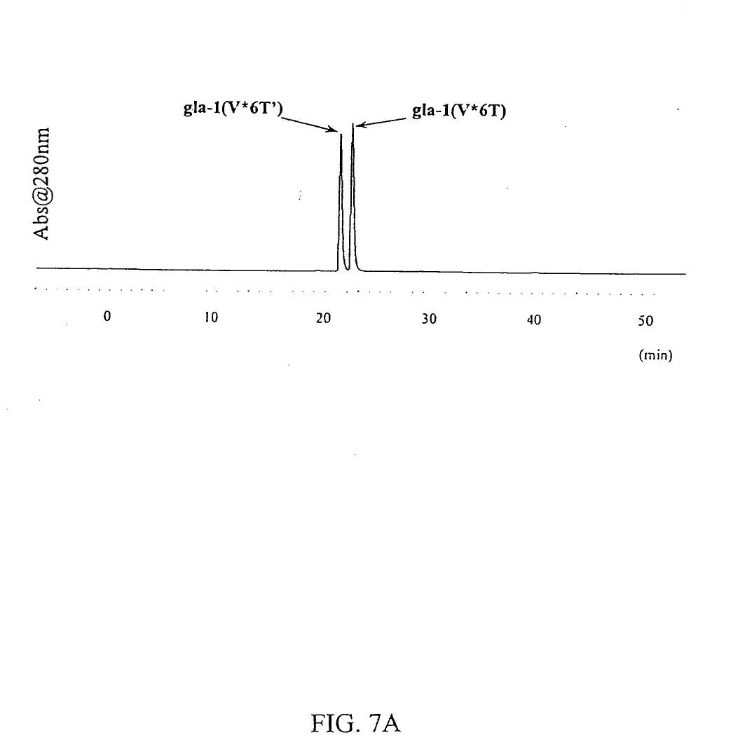

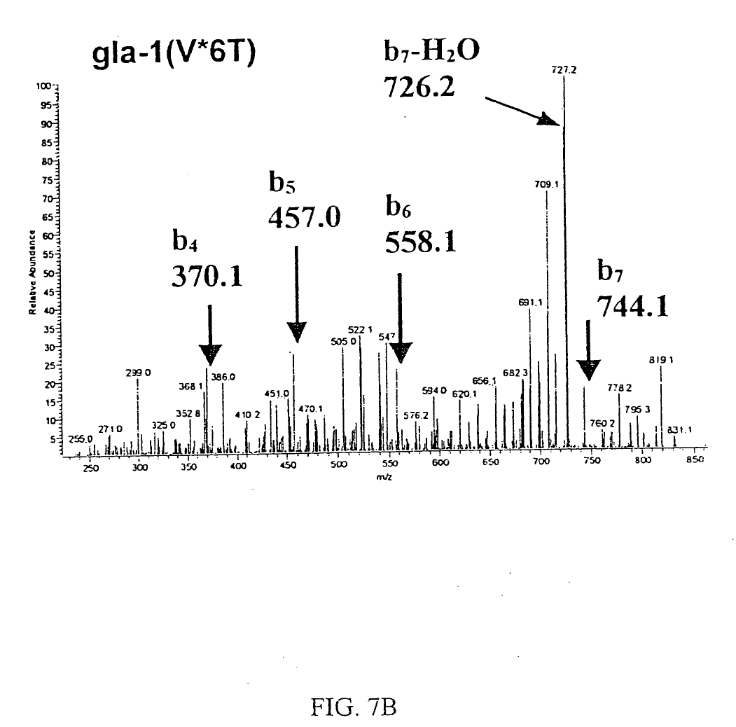

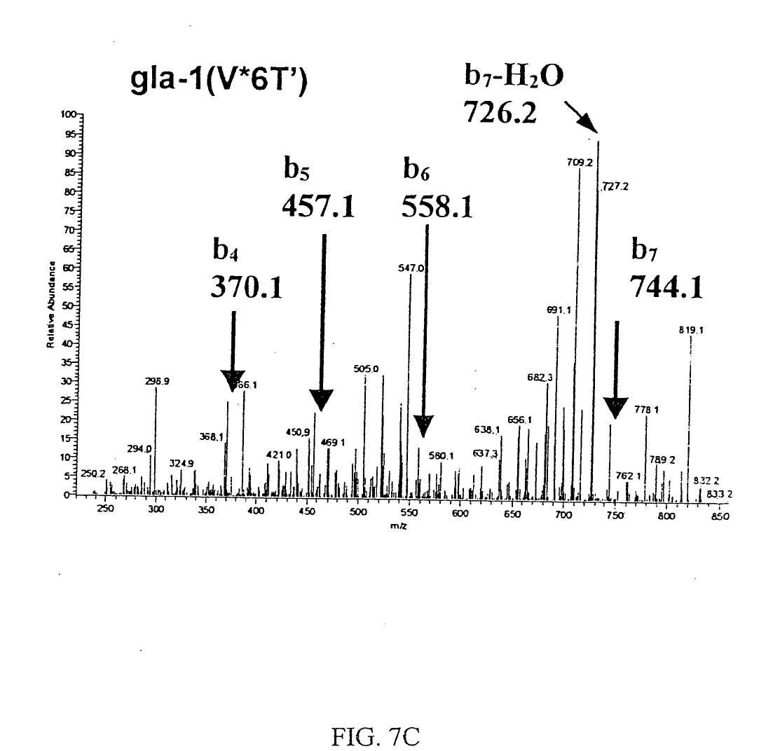

To establish the absolute configuration around the β carbon (C-3) in Hyv of the gla conopeptides, two diastereomeric analogs of gla-1, epimeric at C-3, were synthesized. These synthetic analogs were termed gla-1 (Hyv6Thr) and gla-1 (Hyv6Thr′), where Thr′ is allo-threonine. FIG. 7A shows the RP-HPLC chromatogram of the gla-1(Hyv6Thr) and gla-1(Hyv6Thr′) analogs FIGS. 7B and 7C show the respective MS/MS spectral data for the gla-1 (Hyv6Thr) and gla-1 (Hyv6Thr′) analogs. Direct comparison of the chromatographic and spectrometric behavior of these analogs with known configurations at the chiral centers in residue 6 indicated that gla-1 is likely to correspond to the 2S, 3S configuration in its Hyv residue, whereas gla-1′ corresponds to the 2S, 3R configuration in its Hyv residue. Thus the results with the synthetic peptides of known chirality in residue 6 (Hyv) established that gla-1 and gla-1′ are diastereomeric at Hyv.

### EXAMPLE 12

### Chemical Structure of γ-Hydroxyconophans

From the combined NMR, ESI-MS/MS, and Edman degradation analyses of the gla and mus octa-conopeptides, the following sequences were determined for these peptides:

| | | |
|---|---|---|
| gla-1/gla-1′ | A-O-A-N-S-V*-W-S | (SEQ ID NO: 5) |
| gla-2 | A-O-A-N-S-V-W-S | (SEQ ID NO: 7) |
| mus-1/mus-1′ | A-O-S-N-S-V*-W-S | (SEQ ID NO: 6) |
| mus-2 | A-O-S-N-S-V-W-S | (SEQ ID NO: 8) |

wherein:
O=γ-hydroxyproline;
V*=γ-hydroxyvaline; and
W=D-Trp.

As can be seen from a comparison of SEQ ID NOS:5-8, the gla conophans differ from their mus counterparts at residue 3, by the presence of Ala, as opposed to Ser. Accordingly, a generalized structure for the gla and mus family of octapeptide conopeptides is the following:

| | |
|---|---|
| A-O-X₁-N-S-X₂-W-S | (SEQ ID NO:1) |

wherein:
O is γ-hydroxyproline;
X₁ is A or S;
X₂ is V or γ-hydroxyvaline (V*); and
W is D-Tryptophan.

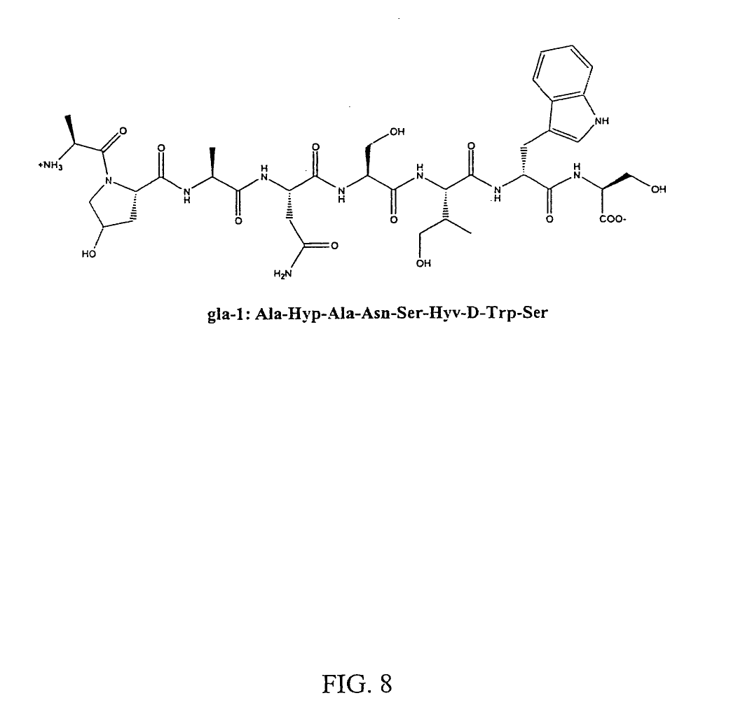

A preferred embodiment of a γ-Hydroxyconophan octapeptide is gla-1 (SEQ ID NO:5), which has the chemical structure shown in FIG. 8.

### EXAMPLE 13

### Molecular Modeling of Hydroxyconophan Structure

Molecular models were built and optimized using the MMX force-field as described (Mari, F., Lahti, P. M., McEwen, W. E. Journal of the American Chemical Society 1992, 114, 813-821). The structural motif that characterizes the gla/mus γ-Hydroxyconophan family of conopeptides [Hyv-D-Trp] was capped using an acetyl group at the N-terminus and an amide group at the C-terminus to simulate a protein-like environment. An extended conformation consistent with the NMR data was used for the initial model and optimized to self-consistency by the force field.

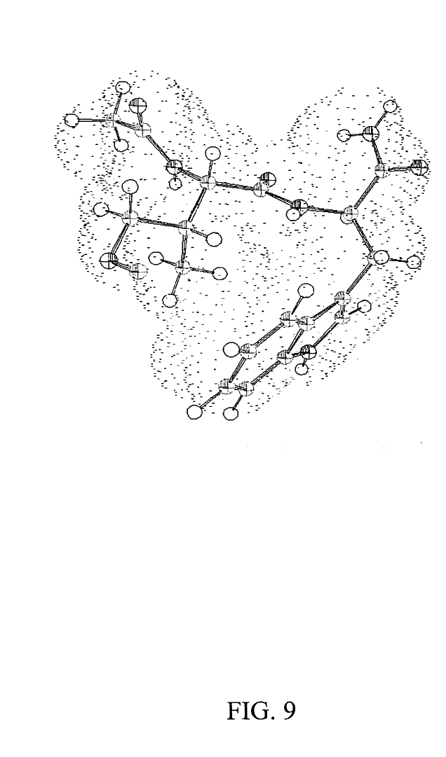

FIG. 9 shows a molecular model of a γ-Hydroxyconophan including the structural motif H₃CC(O)-Hyv-D-Trp-NH₂. This model is consistent with the stabilizing interactions and NMR data described above. It is known that intramolecular lactone formation cannot occur when constrained hydroxylated amino acids such as Hydroxyproline (Hyp) are found in polypeptide chains (Jimenez, E. C., Olivera, B. M., Gray, W. R., Cruz, L. J. Journal of Biological Chemistry 1996, 271, 28002-28005; Jacobsen, R., Jimenez, E. C., Grilley, M., Watkins, M., Hillyard, D., Cruz, L. J., Olivera, B. M. Journal of Peptide Research 1998, 51, 173-179; Pallaghy, P. K., Melnikova, A. P., Jimenez, E. C., Olivera, B. M., Norton, R S. Biochemistry 1999, 38, 11553-11559; Raybaudi Massilia, G., Eliseo, T., Grolleau, F., Lapied, B., Barbier, J, Bournaud, R., Molgo, J., Cicero, D. O., Paci, M., Eugenia Schinina, M., Ascenzi, P., Polticelli, F. Biochemical and Biophysical Research Communications 2003, 303, 238-246). The model indicates that the D-configuration of the Trp residue is required to disfavor the intraresidue cleavage of the peptide bond by the γ-hydroxyl group.

### EXAMPLE 14

### Neuronal Cell Culture System for Testing In Vitro Biological Activity of Conopeptides

Primary cultures of cortex were prepared from cortical hemispheres isolated from anaesthetized neonatal rat pups. Hemispheres were cleaned of meninges and the hippocampus removed and discarded. The cortex was dissociated using 20 U/ml Papain and trypsin with constant mixing for 45 min at 37° C. Digestion was terminated with fraction V BSA (1.5 mg/ml) and Trypsin inhibitor (1.5 mg/ml) in 10 mls media (DMEM/F12+10% fetal Bovine serum and B27 neuronal supplement (Life Technologies). Using gentle trituration, cells were separated from the surrounding connective tissue. Using a fluid-handling robot (Quadra 96, Tomtec) cells were settled onto Poly-D-lysine coated white/clear 96 well plates (Becton-Dickenson). Each well was plated with approximately 25,000 cells. The plates were placed into a humidified 5% CO₂ incubator at 37° C. and kept for at least 7 days before fluorescence screening.

### EXAMPLE 15

### Fluo-3 Assay of Cellular Ca⁺⁺ Flux in Cultured Cortical Neurons

The ability of γ-Hydroxyconophan compounds to modulate flux of Ca⁺⁺ ions in cultured neuronal cells was tested using a 96 well plate fluorimetry protocol utilizing the the Fluo-3 ester calcium dye method. As is well known, this dye becomes effectively trapped within cells following cleavage of the ester from the Fluo-3 dye by esterases within the cell cytoplasm. Cells were incubated for 45 min at 37° C. The Fluo-3 dye (2 μM) was loaded into cells with 20% pluronic acid.

Prior to beginning the experiments, the cells were washed thoroughly with saline solution using a Tomtec liquid handling robot. Initial readings were taken with a cytofluor 4000 HT fluorimeter (Perceptives) set at 36° C. to assess the background fluorescence in each of the plates. The excitation/emission settings were 483/530.

Test compounds were added with a liquid handling robot as described above. All compounds were tested at a concentration 3 μM. Some compounds were screened in both undepolarized and depolarized (10 mM KCl) conditions. For those experiments that required pretreatment, cells were pretreated for 30s with the compound before application of 10 mM KCl. This concentration of KCl was selected based on the results of a KCl concentration-response experiment performed in primary cultures of rat neonatal cortex which demonstrated that a significant and robust increase in intracellular calcium was observed at this concentration. In each plate, a control saline well was included and either a second saline well or a KCl well were included. The saline solution used in the assays contained (in mM) 137 NaCl, 5 KCl, 10 HEPES, 25 Glucose, 3 CaCl₂, and 1 MgCl₂ (brought to pH 7.3 with NaOH). Each compound was run in 4 wells per plate in 3 separate plates. Readings were taken at intervals of 15s, 2 min, 5 min and 10 min.

Data were analyzed using Microsoft Excel and graphed using Origin software (Microcal). The initial background fluorescent readings were subtracted from the treatment readings. The saline control was then subtracted and the results expressed as an absolute change in fluorescence. Increases in intracellular calcium measured with the Fluo-3 dye are reflected as rises in fluorescence. Conversely, decreases in fluorescence represent a drop in intracellular calcium.

### EXAMPLE 16

### Modulation of Neuronal Ca⁺⁺ Flux By Conopeptides

Several γ-Hydroxyconophan and Conophan compounds were tested in the assay described in Example 16 for their ability to modulate cellular Ca⁺⁺ flux in cultured neurons. The results showed that gla-1, gla-1′ and gla-2 at 3 μM concentration all had the effect of reducing Ca⁺⁺ flux in the cells. By contrast, the saline control had the opposite effect, causing an increase in cellular Ca⁺⁺. This result demonstrated that the γ-Hydroxyconophan and Conophan peptides tested were biologically active, modulating the flux of calcium in the cells, presumably through interaction with cell surface receptors controlling calcium levels in the cells.

### EXAMPLE 17

### Novel Conotoxin Framework: an Helix-Loop-Helix (Cs α/α) Motif Peptides

Crude venoms from C. villepinii (113 mg) and C. floridanus floridensis (97 mg) were initially size-fractionated using a HPLC Superdex 30 column. Different absorbance peaks were observed and the column fractions identified by the arrows were purified using reverse phase HPLC with a semipreparative C18 column and further purification to homogeneity was done with an analytical C18 column utilizing conditions described under “Materials and Methods”.

Sequences determination: The purified peaks were subjected to reduction and alkylation. Mass spectrometry of the reduced/carboxymethylated peptides and of native peptides showed a mass difference consistent with the presence of four cysteine residues for each peptide. The native peptides were sequenced by automated Edman degradation and yielded clear residue assignments for all 27 cycles. Peptides flf13a and flf13b only have one amino acid different. The four peptides have a new disulfide framework with an identical separation between the cysteine residues. A Basic Local Alignment Search Tool (BLAST) search (Altschul et al., 1997) of the data base did not show any significant homology to knowing proteins or peptides, indicating that this are structurally novel peptides.

Determination of the molecular mass: Mass spectrometry was carried out using MALDI-TOF in the reflector mode yielded monoisotopic mass signals for each peptide. The masses obtained for the peptides were in a good agreement with the calculated monoisotopic for the assigned sequence and indicated that the peptides were not amidated at the C-terminus.

Disulfide connectivity analysis: The disulfide pattern could not be established with the method of partial reduction and alkylation due to the low concentration of the peptides obtained after the purification. In this case, the four peptides gave a sequence with four separates cysteine residues, this offer the advantage to determine the disulfide connectivity using specific cleaving in between the loops of the peptides without reduction the disulfide bridges in order to yield fragments whose molecular weight will depend of the disulfide bonding. The molecular weights of these fragments could be determined by mass spectrometry with the advantage that required low quantities of sample. Based on the three possibilities of disulfide bonds for these peptides, the use of a combination of CNBr and chymotrypsin could yield different disulfide fragments with different molecular weights. This procedure was applied for peptides flf13a, flf13b and vil13a because the three of them have a methionine residue in the third loop that could be first cleaving by CNBr. Further cleaving of the other loops was achieved by chymotrypsin that cleave aromatic residues like Phe, Trp, Tyr. The calculated molecular weights of some of the predictable fragments for each disulfide bond possibility, except C6-C10; C22-C26 because vicinal disulfide bonds are extremely rare. The MALDI-TOF mass spectra after the treatment of each peptide with CNBr/chymotrypsin combination shows the molecular weights of the disulphide bonded fragments obtained indicating that the disulfide pattern for the three peptides is C6-C26; C10-C22. No fragment that represents the other possible disulfide patterns was found. Some fragments with methionine shows a MH⁺-30 Da that correspond to a homoserine formed from Met by CNBr treatment, in the resulted fragments of vil13a the loss is 60 Da due the present of two residues of Met in the peptide. A product of dehydration (MH⁺-18 Da) in the fragment DVNDCIHF bonded to CT is probably due to an intramolecular condensation reaction between two amino acids of the fragment. Other peaks observed are intermediate products of digestion.

Circular dichroism spectra: CD spectra of flf13a, flf13b, flf13c and vil13a are very similar to each other with a minimum around 208 nm. Peptide vil13a shows other minimum at 225 nm. According to these CD spectra the secondary structure of these peptides is highly α-helical.

NMR Spectroscopy: wgNOESY spectra of flf13a and vil13a at 25° C. It should be noted that in spite of the small size (27 residues), a large number of NOE (>300) cross-correlations were found for these peptides. This feature was also observed flf13b and flf13c peptides. The NMR behavior at room temperature of these peptides is reminiscent to the one observed in larger tightly folded globular proteins. The NMR structures of other two disulfide-bonded conotoxins (α, π and χ) have been determined. However, these determinations have been carried out at lower temperatures and with relative low number of NOE constraints per residue. The four peptides have very well defined structure in solution at room temperature. Highly structured frameworks has been observed in three disulfide-bonded conotoxins belonging to the O and M superfamilies, where a cysteine knot with a triple-stranded β-sheet is the prevalent structural motifs within these tightly compact scaffolds. T his is not the case of the peptides flf13a, flf13b, flf13c and vil13a, since here there are two disulfide bonds and all four cysteine residues are spaced by loops of at least three amino acids. In a good agreement the results of CD spectra and NMR spectrometry revealed a α-helical secondary structure for the four peptides. This is confirmed by the presence of several sequential NOE NN (i to i+1) in conjunction with the αN (i to i+1) in the finger print region. Sequence specific assignments and the full 3D structural determination of vil13a is currently in progress.

The isolation of four novel Conus peptides from the venom of C. villepinii and C. floridanus floridensis revealed these peptides surprisingly differ highly with other conotoxins. A BLAST analysis (Altschul et al., 1997, Nucleic Acids Research, 25, 3389-3402) of the SwissProt databases indicated that the peptides belong to a novel family. The conserved cysteine motif of X₅CX₃CX₁₁CX₃CX₁ was observed in all four peptides; in addition residue Ile7 is conserved. It is interesting that this new motif was found in two different Conus species. We found that even though these peptides have the same framework, the inter-cysteine amino acid sequences are quite variable. All α-conotoxins, T-conotoxins and σ-conotoxin have a Cys1-Cys3, Cys2-Cys4 connectivity. In contrast, α-conotoxins have Cys1-Cys4, Cys2-Cys3 connectivity. Since the peptides have two disulfide bonds and all four cysteine residues are spaced, it allowed the use of specific cleaving in between the loops. The advantage of this method is that it requires low quantities of sample and the fragmentation products can be directly detected by MALDI-TOF.

Conotoxins can bind and discriminate closely related targets. The loop spacing and composition are important for the target specificity. The results of CD spectra and NMR spectrometry revealed an α-helical secondary structure for the four peptides. Using a web-based application tool named CysView (Lenffer et al, 2004, Nucleic Acids Research, Vol. 32. W350-W35) that identifies and classifies proteins according to their disulfide connectivity patterns we found several proteins and peptides with the cysteine pattern (1,4)(2,3).

It is surprising that peptides from two Conus species were similar to a scorpion toxin. Some potassium channel toxins have the presence of a functional diad that resides in a critical lysine and an aromatic residue separated by 6.0±1.0 Å. This diad was found in structurally unrelated potassium channel-blocking toxins from scorpions (Miller et al., 1985, Nature 313, 316-318) and sea anemones (Castafieda et al., 1995, Toxicon. 33, 603-613).

### EXAMPLE 18

### Hyperhydroxylation: A New Strategy for Neuronal Targeting by Venomous Marine Molluscs

To analyze and characterize conopeptides isolated from cone snail species from the Americas, it was decided to undertake the isolation and structural analysis of conopeptides from Conus regius (species code: reg), a widespread worm-hunting cone snail of the Western Atlantic Ocean. Results from the isolation and structural analysis of three hydroxylated conopeptide families from Conus regius are presented: α-conotoxins, the newly described mini-M conotoxins and a novel family of linear conopeptides that we have termed 6γ. These peptides exhibit a differential or preferential proline hydroxylation strategy that is likely to affect their neuronal targeting. Beyond the hydroxylation of Pro, we have discovered a new family of conopeptides that feature the modified amino acid D-γ-hydroxyvaline (Hyv=V*). This doubly modified amino acid is part a novel structural motif, Ser-D-γ-OH-Xaa-Trp, that defines a new class of conopeptides that are termed, herewith, γ-hydroxyconophans. Hydroxyconophans constitute the first examples of a polypeptide chains containing Hyv. We have also isolated analogous conopeptides comprising D-Val instead of D-Hyv; these are termed conophans. Additionally, we have isolated three related conopeptides from Conus vilepinii (vil-M, vil-I and vil-I(O2P) that are similar in sequence and properties to the gla/mus conophans; however, vil-M and vil-I incorporate D-Ile and D-Met instead of D-Val.

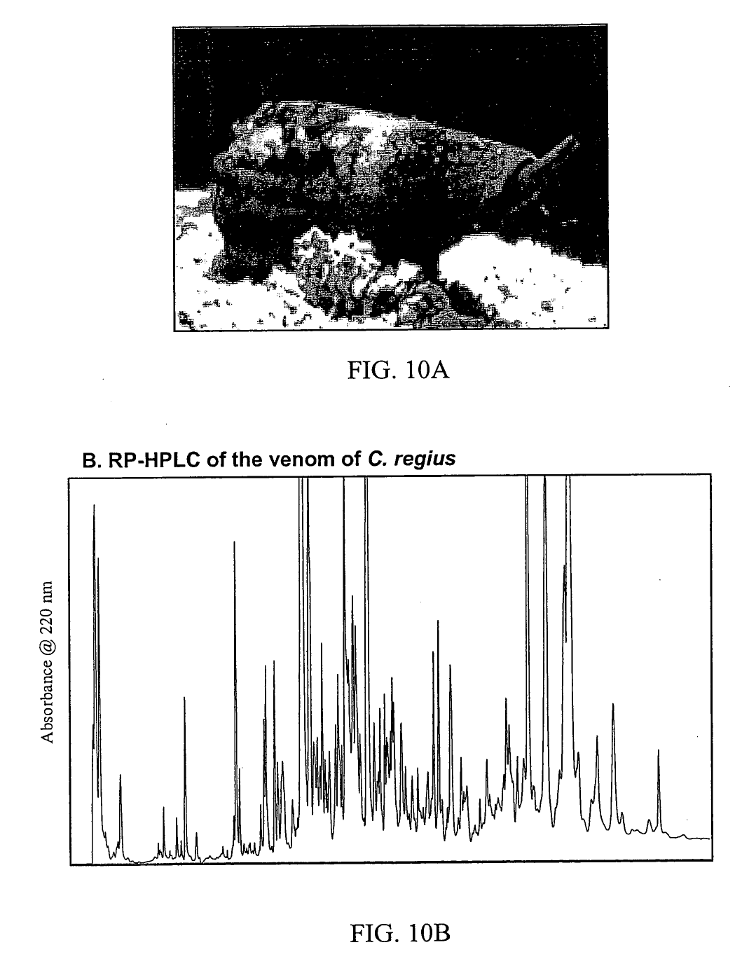

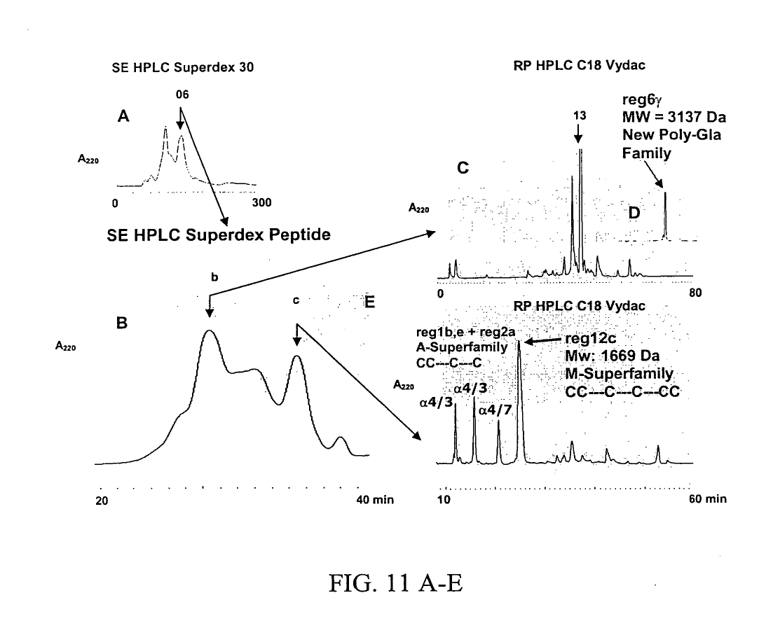

Hydroxylation of α-conotoxins: The venom of Conus regius is an extremely complex mixture of peptides and proteins whose direct separation is shown in FIG. 10. More than 100 fractions can be obtained from this separation. However, most of these fractions show multiple components, for which it was required to adopt an improved separation scheme that includes a prefractionation step using size exclusion chromatography on a Superdex-30 column, followed by a refined peptide-optimized size exclusion step on a Superdex Peptide column. The resulting fractions were then separated by reversed phase on a peptide-optimized C18 Vydac Everest column. Most of the resulting fractions were single-component (FIG. 11) and were subsequently analyzed by mass spectrometry (MALDI-TOF and ESI-Q-TOF), NMR spectroscopy and o peptide sequencing by Edman degradation chemistry. Using this methodology, the complete analysis of the most significant components of the venom of C. regius can be sequenced (Conopeptidome) and the components of the venom can be grouped in the different families of conopeptides. Three predominant families of hydroxylated conopeptides were found in the venom of Conus regius: α-conotoxins (4/3 subtype), mini-M conotoxins and a novel family of poly-Gla containing linear conopeptides that we have termed 6γ.

TABLE 1
a-Conotoxins from Conus regius

| | | |
|---|---|---|
| | | Target: |
| a-4/3 | Sequence | nAChR |

| | | | |
|---|---|---|---|
| reg1a | --GCCSDORCRYX----C* | (SEQ ID NO.: 9) | |
| reg1b | --GCCSDORCKHQ----C* | (SEQ ID NO.: 10) | |
| reg1c | --GCCSDPRCKHQ----C* | (SEQ ID NO.: 11) | |
| reg1d | --GCCSDPRCKHE----C* | (SEQ ID NO.: 12) | |
| reg1e | --GCCSDORCRYR----C* | (SEQ ID NO.: 13) | |
| reg1f | -DYCCRROOCTLI----C* | (SEQ ID NO.: 14) | |
| ImI | --ACCSDPRCAWR----C* | (SEQ ID NO.: 15) | a7 |
| ImII | --GCCSDRRCAWR----C* | (SEQ ID NO.: 16) | a7 |
| a-4/7 | | | |
| reg2a | --GCCSHPACNVNNPHIC* | (SEQ ID NO.: 17) | |
| GID | RDγCCSNPACRVNNOHVC | (SEQ ID NO.: 18) | α3β2,a7 >α4β2 |
| GIC | --GCCSHPACAGNNQHIC* | (SEQ ID NO.: 19) | α3β2 |
| Vc1a | GCCSDORCNYDHPγIC* | (SEQ ID NO.: 20) | α3α7β4/α3α5β4 |
| AnIC | GGCCSHPACAANNQDŶC* | (SEQ ID NO.: 21) | α3β2,α7 |
| EI | RDOCCYHPTCNMSNPQIC* | (SEQ ID NO.: 22) | α1γ = α1δβ2 musc. |

Table 1 shows the sequences of the α-conotoxins found in the venom of C. regius. Six of these α-conotoxins belong to the 4/3 subtype and one of the 4/7 subtype. α4/3 subtypes are rare within the α-conotoxin family. reg1a-f are the only α4/3-conotoxins isolated from an Atlantic cone snail species with known sequences to date. The reg1a,b,e and f conotoxins are also unusual as they are the only α4/3-conotoxins whose sequences are post-translationally hydroxylated to produce the modified amino acid hydroxyproline: reg1f has two hydroxyproline residues in its first loop, whereas reg1a, reg1b and reg1e have one Hyp. reg1b and reg1c have the same sequence except that reg1d is not hydroxylated at Pro-6. reg1c and reg1d only differ in residue 11 (Gln in reg1d vs. Glu in reg1d). reg1a-e have significant sequence homology; they share the same sequence in the first loop (SDPR or SDOR) and a conserved positively charge residue (Arg or Lys) in the first position of the second loop. reg1a-e are homologous with the only other known α4/3 conotoxins, ImI and ImII; these two toxins only differ in one residue (Hyp6 in 1 ml, Arg6 in ImII) and they both bind to the α7 subtype neuronal nAChR. However, these two conotoxins bind at different sites and their mechanisms of inhibition appear to be different in spite of their sequence homology. Likewise, the reg1a-e conotoxins are likely to show differential binding properties towards the nAChR. By way of contrast, reg1f has no sequence homology to the other known α4/3 conotoxins; as its sequence (i) has an extended N-terminal tail (ii) lacks of a the conserved Ser in the first residue of loop 1 seen in virtually all α4/3 and 4/7 conotoxins; Arg residue is in its place (iii) first loop (RROO) has no homology with the other α4/3 conotoxins; only the first Hyp with other α4/3 and 4/7 conotoxins and (iv) second loop (TLI) has no homology with the other α4/3 conotoxins and notably lack positively charged residues. Presumably, this departure from the sequences of other α-conotoxins has profound consequences to its binding to the neuronal nAChR. All these conotoxins are either hydroxylated or susceptible to hydroxylation, which is a feature relatively rare among α-conotoxins (Dutton and Craik 2001, Current Medicinal Chemistry 8:327-344; Livett et al. 2004, Current Medicinal Chemistry 11:1715-1723; Millard et al. 2004, Eur J Biochem 271:2320-2326; Nicke et al. 2004, Eur J of Biochem 271:2305-2319). These modifications enhance the polarity and hydrogen-bonding capabilities to these conotoxins and are likely to define their mode of binding to the nAChR.

In addition to the α4/3 conotoxins, an α4/7 was found in the venom of Conus regius, reg2a. This conotoxin, while not hydroxylated, shows extraordinary homology with GIC (J Biol Chem. 277:33610-33615) and GID (J Biol. Chem. 278:3137-3144), a pair of α4/7-conotoxins isolated from Conus geographus, the quintessential Indo-Pacific fish-hunting cone snail species. This similarity in sequence is quite remarkable considering that C. regius is a worm-hunting cone snail from the Western Atlantic. However, C. geographus does not contain α4/3 conotoxins within its venom. Perhaps, the proper combination of α-conotoxins subtypes is required for the effective targeting the neuronal receptors in order to produce a synergistic disabling effect on the different prey to be captured.

Hydroxylation of mini-M conotoxins: Within the venom of Conus regius, another prevalent family of conopeptides is the mini-M or M2 subclass of conotoxins that belong to the M-Superfamily (McDougal and Poulter 2004, Biochemistry 43:425-429). Mini-M conotoxins share the same arrangement of Cys in their sequence as other members of the M-superfamily (CC-C-C-CC), such as the Maxi-M or M1 subclass. The loops in the Mini-M are much shorter and the Cys pairing is different from the classical Cys knot observed in the Maxi-M subclass. Several targets have been identified in the Maxi-M subclass: voltage gated sodium channels (μ-Conotoxins), voltage gated potassium channels (κM-Conotoxins) and the nAChR (ψ-Conotoxins). Invariably, all of these conotoxins are fully hydroxylated at their Pro residues. Mini-M conotoxin molecular targets have not been identified; however, it appears to be different from the maxi-M families.

TABLE 2
mini-M-Conotoxins from Conus regius

| | | | |
|---|---|---|---|
| Mini-M (m-2) | | | |
| reg12a | GCCOOQWCGOG--CTSOCC | (SEQ ID NO.: 23) | 4/3/3 |
| reg12b | LCCOOQXCGOD--CASOCC | (SEQ ID NO.: 24) | 4/3/3 |
| reg12c | -CCTAL-CSRYH-CL-PCC | (SEQ ID NO.: 25) | 3/4/2 |
| reg12d | KCCMRPICT----C--OCCIG | (SEQ ID NO.: 26) | 4/1/1 |
| reg12e | GCCPFPACTHHIICR--CC | (SEQ ID NO.: 27) | 4/5/1 |
| reg12f | GCCSOWNCIQLRAC--OCCON | (SEQ ID NO.: 28) | 4/5/1 |
| reg12g | KCCMRPICM----C--OCC | (SEQ ID NO.: 29) | 4/1/1 |
| reg12h | RCCPMPGCFAGPFC--PCC | (SEQ ID NO.: 30) | 4/5/1 |
| Mr3a | 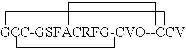 | (SEQ ID NO.: 31) | 4/3/2 |
| RIIIK (κM Cntx) | 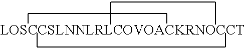 | (SEQ ID NO.: 32) | 7/4/4 |
| from cDNA of C. radiatus | | | |
| Shaker K+ channel blocker | | | |
| maxi-M (m-4) | | | |
| μGIIIA C. geographus | RDCCTOOKKCKDRQCKOQRCCA | (SEQ ID NO.: 33) | |
| 5/4/4 μPIIIA C. purpurascens | RLCCGFOKSCRSRQCKOHRCC | (SEQ ID NO.: 34) | 5/4/4 |
| ψPIIIE C. purpurascens | HOCCLYGK-CRRYOGCSSASASCCQR | (SEQ ID NO.: 35) | 4/5/6 |
| non comp. nAChR | | | |

To date, all reported conotoxins of the M-superfamily are hydroxylated at the Pro residues of their sequences. In fact, it has been assumed to be a conserved feature of this superfamily. We isolated seven mini-M conotoxins from the venom of C. regius: reg12a-f (Table 2). Unlike the reg1 α-conotoxins, there is no sequence homology among the reg12 mini-M conotoxins. When considering the size of their loops (a defining feature within the M-Superfamily), there are four variants within the reg12 conotoxins: 3/4/2, 4/1/1, 4/3/3 and the 4/5/1 subtypes. Just like the other members of the M-superfamily, reg12a, reg12b and reg12f are hydroxylated at all Pro residues of their sequences, just as the known maxi-M conotoxins. Surprisingly, the Pro residues of reg12c, reg12e and reg12e are unmodified; notably the latter did not exhibit hydroxylation in spite of having four Pro residues with its sequence. reg12d and reg12g are partially hydroxylated as one of their two prolines were found to be a Hyp residue. The sequence diversity found within these reg12 mini-M conotoxins suggests that their targeting of neuronal receptors and/or ion channels might be equally diverse. Perhaps in this case hydroxylation is part of a refinement strategy, where certain sequences within this family are required to have the additional polarity and hydrogen bonding capability, whereas others do not. Differential hydroxylation has been observed within the same conopeptide sequence (Hopkins C, et al., (1995) J Biol Chem. 270:22361-22367); this does not appear the case for the reg12 mini-M conotoxins.

6γ: a novel family of polycarboxylated and polyhydroxylated conopeptides: In addition to the a and mini-M conotoxins, another major component of the venom of C. regius is a novel class of linear conopeptides that we have termed 6γ. reg6γ is a polycarboxylated (at Glu residues) and polyhydroxylated (at Pro residues) glycopeptide whose complete sequence is shown in Table 3. In Table 3 the sequences of the novel reg6γ glycopeptide was compare with other polycarboxylated conopeptides. The most distinct feature of reg6γ is the presence of six Gla residues. Polycarboxylated conopeptides are known, and it is the main characteristic displayed by Conantokins, a class of linear conopeptides that have 4-5 Gla residues. These compounds are characterized by the presence of a Gly-Glu-Gla-Gla N-terminus and a Gla repeat that is quite different from the one observed in reg6γ (Table 3). Unlike Conantokins, reg6γ has three hydroxylated proline residues (Hyp). Polyhydroxylation is a standard feature for some conopeptide families, such as the 1′-conotoxins, ψ-conotoxins and the κA-conotoxins; however, it has not been observed in conjunction with polycarboxylation. Additionally, reg6γ appears to be glycosylated. Glycosylation, while not a recurrent modification in most conopeptide families, can be found in Contulakins (as the major modification of these linear conopeptides), some κA conotoxins (κA-SIA and κA-CnIVA), ε-conotoxins (T-superfamily) and in other undefined conopeptides. reg6γ is the product of polycarboxylation, polyhydroxylation and glycosylation operating in a unique sequence (no matches in the sequence databases were found of the unmodified peptide sequence) to produce a new family of conopeptides: 6γ.

TABLE 3
Linear polycarboxylated conopeptides

| | | |
|---|---|---|
| Family | Sequence | Conus Species |

| | | | |
|---|---|---|---|
| 6γ | | | |
| reg6γ | RVLγOGXγDODVGγOAGγYγHHLLγX | (SEQ ID NO.: 36) | C. regius |
| Conantokin(3-5γ) | | | |
| Con G | GEγγQγNQ-γLIRγKSN | (SEQ ID NO.: 37) | C. geographus |
| Con T | GEγγQKML-γNLRγAEVKKNA | (SEQ ID NO.: 38) | C. tulipa |
| Con R | GEγγAKMAAγLARγNIAKGCKVNCYP | (SEQ ID NO.: 39) | C. radiatus |
| Con L | GEγγAKMAAγLARγDAVN | (SEQ ID NO.: 40) | C. lynceus |
| Con S1 | GDγγSKFI-γRERγAGRLDLSKFP | (SEQ ID NO.: 41) | C. sulcatus |
| Con Qu | GYγγ-RγVAγTVRγLDAA | (SEQ ID NO.: 42) | C. quercinus |
| Con Ca2 | GYγγ-RγIAγTVRγLEEA | (SEQ ID NO.: 43) | C. caracteristicus |
| Con Gm | GAKγ-RNNAγAVRγRLEEI | (SEQ ID NO.: 44) | C. gloriamaris |
| Con Oc | GEγγRKAMAγLEAKKAQγALKA | (SEQ ID NO.: 45) | C. orchroleucus |

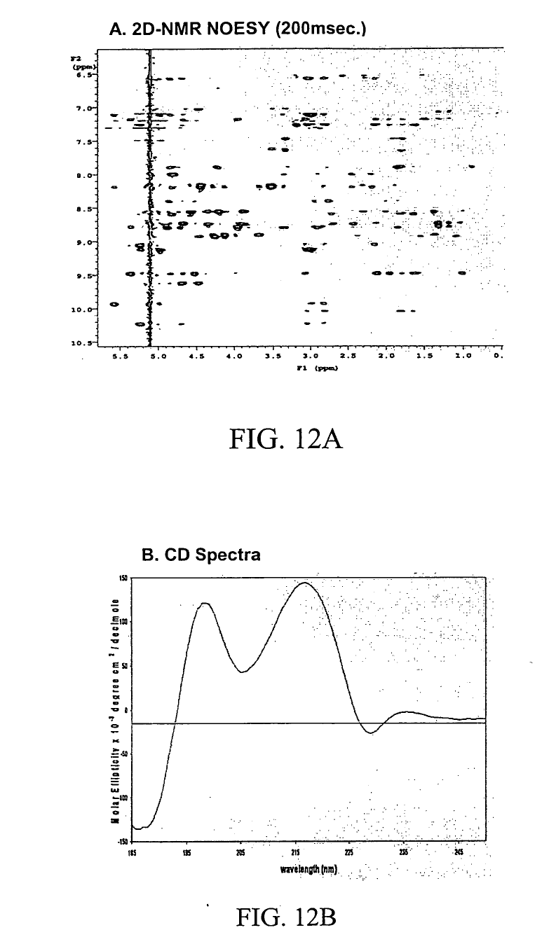

The structural features of the novel reg6γ conopeptide were investigated by 2D-NMR and CD spectroscopic methods methods. reg6γ was structured at 25° C. (as judged by the number of NOEs in the wgNOESY spectra), in spite of its lack of disulfide bond constrains. Several strong HN to HN and αH to HN correlations were observed in the NOESY spectra (FIG. 12A) suggesting that reg6γ might have a helical structure just like the conantokins. However, the CD spectra of reg6γ (FIG. 12B) shows an unusual trace characterized by signals of positive ellipticity. Similar CD spectra have been observed in proteins of high β-sheet content; this appears to contradict the NMR findings. Nevertheless, reg6γ is quite a unique scaffold, where known modifications within Conus biochemistry are combined to produce a highly stable and unusual conopeptide.

All conopeptides found in Conus regius discussed above represent novel sequences that exhibit different degrees of hydroxylation within their frameworks: the first set of hydroxylated α4/3-conotoxins, a set of mini-M conotoxins that ranges from fully hydroxylated sequences to non-hydroxylated ones and a new polycarboxylated conopeptide family that is also fully hydroxylated and defines a highly stable structural framework without cysteine bridges.

Hyperhydroxylation of Conophans: D-γ-hydroxyvaline and γ-hydroxyconophans: We have described a new family of linear short conopeptides that we have termed γ-hydroxyconophans. These conopeptides have a high content of hydroxyl residues: Ser, Hyp and the unprecedented presence of D-γ-hydroxyvaline in their sequence. These conopeptides are the first examples of polypeptides chains containing such an intriguing modification. Four novel conopeptides were characterized from the venom of Conus gladiator (gld-V* and gld-V*′) and Conus mus (mus-V* and mus-V*′). These conopeptides contain the doubly modified amino acid D-γ-Hyv. Their respective peptidyl precursors were also characterized, that contain D-Val (gld-V and mus-V).

We initially isolated three unusual conopeptides, gld-V*, gld-V*′ and gld-V, from the venom of Conus gladiator (species code gld), a cone snail species that inhabits the tropical Eastern Pacific region and preys upon worms. Upon the identification of these conopeptides, related conopeptides mus-V*, mus-V*′ and mus-V were isolated from Conus mus (species code mus), a cone snail species related to C. gladiator that inhabits the Western Atlantic region. C. gladiator and C. mus share an ancestral origin that split 3 mbp upon the raise of the Isthmus of Panama. The gld conopeptides were isolated in nanomolar quantities, whereas the mus conopeptides were isolated in picomolar quantities. Nano/pico-NMR techniques allowed the acquisition of their spectra and revealed almost identical compositions for these octapeptides, including an unusual amino acid for gld-V* and gld-V*′, whereas gld-V showed Val in its place. The mass spectra of gld-V*/gld-V*′ and gld-V gave molecular ions of 863.3 Da and 847.3 Da, respectively. gld-V* and gld-V*′ have the same covalent structures. The mus octapeptides gave information identical to their gla counterparts, except that their MWs were shifted by 16 Da. Combined Edman degradation sequencing, MS/MS and NMR analyses revealed the sequences shown in Table 4.

TABLE 4
Hydroxyconophans and Conophan Library

| | | | |
|---|---|---|---|
| mus-V* | SOANSV*WS | (SEQ ID NO.: 46) | V* = y-Hydroxyvaline |
| mus-V*′ | SOANSV*WS | (SEQ ID NO.: 47) | O = Hyp |
| mus-V | SOANSV WS | (SEQ ID NO.: 48) | V = D-Valine |
| gla-V* | AOANSV*WS | (SEQ ID NO.: 49) | |
| gla-V*′ | AOANSV*WS | (SEQ ID NO.: 50) | |
| gla-V | AOANSV WS | (SEQ ID NO.: 51) | |
| vil-M | EO-NSM WS | (SEQ ID NO.: 52) | M = D-Methionine |
| vil-I | EO-NSI WS | (SEQ ID NO.: 53) | I = D-Isoleucine |
| vil-I(O2P) | EP-NSI WS | (SEQ ID NO.: 54) | |

The D configuration of the Val-6α carbon in gld-V* and mus-V was determined by comparing the chromatographic profiles, NMR and MS/MS spectra of gla-V with synthetic analogs. D-amino acids are known in contryphans; however, here the situation is significantly different, as D-Val has never been found in ribosomally-expressed polypeptide chains and its location (near the C-terminal and in close contact with L-Trp) has important structural connotations not associated with contryphans. The configuration of the α carbon in Hyv was determined by NMR analysis, revealing that its stereochemistry had been preserved upon hydroxylation.

The Ser-D-γ-Hyv-Trp triad is an unusually stable structural motif that incorporates the unprecedented modification of a D-amino acid and has produced the first examples of hydroxyvaline within polypeptide chains. This motif defines the new class of conopeptides: γ-hydroxyconophans. The corresponding precursors, such as gld-V, are termed conophans. The presence of γ-hydroxyvaline in gld-V* as opposed to valine in gld-V suggests the existence of a corresponding enzyme capable of D-Val oxidation. This putative enzyme could be using gla-V, or its precursor protein, as a substrate to modify the specified D-Val to generate the final form of the toxin. This process challenges previous understanding of homochirality in living organisms, as all known enzymatic reactions acting on peptides and proteins are stereoselective for the L-amino acids within them (Petsko G. A., 1992, Science 256:1403-1404).

This unusual transformation appears to be part of a hyperhydroxylation strategy used by cone snails to optimise their venom efficacy by increasing the hydrogen-bonding capabilities of the toxin (see below). In addition to the three Ser residues (for mus-V*′), the hydroxylation of Pro and then D-Val yields a 63% content of hydroxylated residues. The relative stability of the hydroxyconophan may be attributed to specific interaction with the Trp residue that follows the D-γ-hydroxyvaline by CH, π interactions and with the Ser residue that precedes the D-Hyv by hydrogen bonding. NMR evidence and molecular modelling of gld-V* suggest that the D configuration of the Hyv residue allows the stabilizing interaction with Trp and Ser, therefore disfavoring intraresidue cleavage of the peptide bond by the γ-hydroxyl group.

The epimerization and subsequent hydroxylation of Val provides further diversity to the venom by adding a new protein scaffold that is in so far unique to Conus. In addition to the unprecedented presence of D-Hyv in their sequence, γ-hydroxyconophans are unusual because (i) they are linear conopeptides and not constrained like the conotoxin and contryphan families, (ii) they are extremely short in length, (iii) they have a high content of hydroxylated residues, and (iv) their primary structure has no close match in the sequence databases.

In the conophan family, the motif that is defining is the Ser-D-Xaa-Trp triad; where for the conophans of C. gladiator and C. mus the D-amino acid is Val. We have recently isolated three conophans from the venom of Conus vilepinii (species code vil), a Western Atlantic deep-water cone snail species. The sequences of these three conophans, vil-I, vil-I(O2P) and vil-M are shown in Table 4. Their characterization was analogous to the gld and mus conophans.

The vil conophans are among the major components of the venom of C. vilepinii; however, unlike in the case of C. gladiator and C. mus, γ-hydroxyconophans were not found in C. vilepinii. The vil conophans indicate that epimerization is not restricted to Val. Ile and Met can undergo epimerization as well, presumably by the same epimerizase (or an isotype) operating in the case of Val. The presence of just conophans in the venom of C. vilepinii; indicates that this family of conopeptides has a neurochemical function within the arsenal of toxins of cone snails. However, this particular species is using “less hydroxylated” toxins compared to their γ-hydroxyconophans counterparts found in C. gladiator and C. mus, as they have one less Ser residue (in position 3; Table 4) and neither the D-Ile or the D-Met are hydroxylated. This case is reminiscent of the one observed in the α-conotoxins discussed above, where in some instances, cone snails utilize the hydroxylated toxins, in others the non-hydroxylated ones and other times a mixture of both is utilized. Within this scheme of differential hydroxylation, the sequence of vil-I(O2P) suggest a hierarchical sequence of modifications that lead to the hyperhydroxylation observed in the γ-hydroxyconophan family. vil-I(O2P) has a Pro residue in position 2, instead of the Hyp observed in the rest of conophans and hydroxyconophans found so far. This indicates that epimerization, in this case of Ile, precedes hydroxylation suggesting that the sequence of events that lead to hyperhydroxylation proceed as follows: epimerization of the Xaa within the Ser-Xaa-Trp triad, followed by hydroxylation of Pro2 and finally hydroxylation of D-Xaa.

The epimerization of Val, Ile and Met by cone snails has produced the first examples of D-Val, D-Ile and D-Met within ribosomally-expressed polypeptide chains. Most epimerizations found in small linear peptides occur near the N-terminal and preferentially at the second position. The D-Val in these conophans is at the third amino acid from the C-terminal, the same relative position as in the larger disulphide-constrained ω-agatoxin and the r11a I-superfamily conotoxin, which have 48 and 46 residues, respectively. Apparently, the epimerization has a strong preference at this position near the C-terminal regardless of the nature of the amino acid (D-Ser in ω-agatoxin, D-Phe in the r11a conotoxin or D-Val, D-Ile and D-Met in Conophans), or size and nature of the expressed protein. The two-base enzymatic mechanism proposed for the epimerization of D-Ser in ω-agatoxin should be in effect in all these cases, as the epimerase in the funnel web spider epimerizes other amino acids, such as Ala, Cys and O-methylserine. However, the substrate for this epimerase has a recognition site Leu-Xaa-Phe-Ala, observed neither in the r11 conotoxin nor in the Conophans. Furthermore, the spider epimerase is capable of converting Xaa in small peptides at several positions within the polypeptide chain. Therefore, it may be that different epimerases with distinct specificities are operating in each of these cases.

As discussed above, epimerization it is important for hyperhydroxylation. Once epimerization occurs, differential hydroxylation, already described in other conopeptide families is observed. In Conus vilepinii, modification of Pro2 lead to mixtures of vil-I(O2P) and vil-I within the venom, which are involved in the differential neuronal targeting through hydroxylation. In Conus gladiator and mus, modification of D-Val6 leads to mixtures of gld/mus-V* and gld/mus-V*/V*′ in the venom, which are responsible for targeting.

The presence of D-Hyv in gld-V*/gld-V*′, as opposed to D-Val in gld-V, suggests the existence of an enzyme capable of D-Val oxidation. This putative enzyme could be using gld-V, or its precursor protein, as a substrate to modify D-Val and generate the D-Hyv form of the toxin. This process would be analogous to Glu γ-carboxylation of certain conopeptides, which requires the action of a specific carboxylase on the precursor form of the peptide to produce Conantokins, 6γ and related Gla-containing conopeptides. The isolation and identification of a hydroxylase with D-amino acid specificity is under investigation. The function of this enzyme appears to be the augmentation of molecular functionality to enhance neuronal targeting and it is part of a novel neurochemical strategy used by cone snails to capture their prey.

### Other Embodiments

This description has been by way of example of how the compositions and methods of the invention can be made and carried out. Various details may be modified in arriving at the other detailed embodiments, and many of these embodiments will come within the scope of the invention. Therefore, to apprise the public of the scope of the invention and the embodiments covered by the invention, the following claims are made.

What is claimed is:

1. An isolated conopeptide, comprising modified D-valine and proline residues.

2. The isolated conopeptide of claim 1, wherein the D-valine and proline residues are modified by hydroxylation.

3. The isolated conopeptide of claim 1, wherein the conopeptide comprises a triad motif identified by Ser-D-γ-Hyv-Trp and the valine is in a D chiral configuration.

4. The isolated conopeptide of claim 1, wherein at least one D-Val in the conopeptide is hydroxylated.

5. The isolated conopeptide of claim 1, wherein at least two D-Val residues in the conopeptide are hydroxylated.

6. The isolated conopeptide of claim 1, wherein each D-Val residue is hydroxylated.

7. The isolated conopeptide of claim 1, wherein the conopeptide is identified by any one of SEQ ID NO's.: 1-54.

8. A composition comprising an isolated conopeptide, wherein the isolated conopeptide comprises modified valine and proline residues in a pharmaceutically acceptable carrier.

9. The composition of claim 8, wherein the valine and proline residues are modified by hydroxylation.

10. The composition of claim 8, wherein the conopeptide comprises a triad motif identified by Ser-D-γ-Hyv-Trp.

11. The composition of claim 8, wherein at least one D-Val in the conopeptide is hydroxylated.

12. The composition of claim 8, wherein at least two D-Val residues in the conopeptide are hydroxylated.

13. The composition of claim 8, wherein each D-Val residue in the conopeptides are hydroxylated.

14. The composition of claim 8, wherein the conopeptide is identified by an one of SEQ ID NO's.: 1-54.

15. An isolated conopeptide comprising any one of SEQ ID NO's.: 1-54.

16. A composition comprising any one of SEQ ID NO's.: 1-54 in a pharmaceutical carrier.

17. A method for inducing analgesia in a mammal which comprises administering a therapeutically effective amount of a conopeptide identified by an one of SEQ ID NO's.: 1-54.

18. The method of claim 17, wherein said administration comprises using a delivery means selected from the group consisting of a pump, microencapsulation, a continuous release polymer implant, microencapsulation, naked or unencapsulated cell grafts, injection and oral administration.

19. The method of claim 17, wherein the amount of conopeptide administered is between about 0.001 mg/kg to about 250 mg/kg.

20. A pharmaceutical composition comprising a therapeutically effective amount of a conopeptide identified by any one of SEQ ID NO's.: 1-54 or a pharmaceutically acceptable salt or solvate thereof and a pharmaceutically acceptable carrier.

21. The composition of claim 20, which further comprises one or more drugs useful in the treatment of pain.


SEQUENCE LISTING (55 sequences)

SEQ ID NO: 1 - protein, 8 aa, Conus gloriamaris
  x (2)..(2): gamma-hydroxyproline
AXXNSXWS

SEQ ID NO: 2 - protein, 8 aa, Conus gloriamaris
APANSVWS

SEQ ID NO: 3 - protein, 8 aa, Conus mus
APSNSVWS

SEQ ID NO: 4 - protein, 8 aa, Unknown
  genus Conus
AXXNSVWS

SEQ ID NO: 5 - protein, 8 aa, Conus gladiator
  x (2)..(2): gamma-hydroxyproline
AXANSVWS

SEQ ID NO: 6 - protein, 8 aa, Conus mus
  x (2)..(2): gamma-hydroxyproline
AXSNSVWS

SEQ ID NO: 7 - protein, 8 aa, Conus gladiator
  x (2)..(2): gamma-hydroxyproline
AXANSVWS

SEQ ID NO: 8 - protein, 8 aa, Conus mus
  x (2)..(2): gamma-hydroxyproline
AXSNSVWS

SEQ ID NO: 9 - protein, 11 aa, Conus regius
  x (6)..(6): gamma-hydroxyproline
GCCSDXRCRYX

SEQ ID NO: 10 - protein, 12 aa, Conus regius
  x (6)..(6): gamma-hydroxyproline
GCCSDXRCKHQC

SEQ ID NO: 11 - protein, 12 aa, Conus regius
GCCSDPRCKHQC

SEQ ID NO: 12 - protein, 12 aa, Conus regius
GCCSDPRCKHEC

SEQ ID NO: 13 - protein, 12 aa, Conus regius
  x (6)..(6): gamma-hydroxyproline
GCCSDXRCRYRC

SEQ ID NO: 14 - protein, 13 aa, Conus regius
  x (7)..(8): gamma-hydroxyproline
DYCCRRXXCTLIC

SEQ ID NO: 15 - protein, 12 aa, Conus regius
ACCSDPRCAWRC

SEQ ID NO: 16 - protein, 12 aa, Conus regius
GCCSDRRCAWRC

SEQ ID NO: 17 - protein, 16 aa, Conus regius
GCCSHPACNVNNPHIC

SEQ ID NO: 18 - protein, 17 aa, Conus regius
  x (3)..(3): gamma-hydroxycysteine
RDXCSNPACRVNNXHVC

SEQ ID NO: 19 - protein, 16 aa, Conus regius
GCCSHPACAGNNQHIC

SEQ ID NO: 20 - protein, 15 aa, Conus regius
  x (6)..(6): gamma-hydroxyproline
GCCSDXRCNYDHPXC

SEQ ID NO: 21 - protein, 17 aa, Conus regius
GGCCSHPACAANNQDYC

SEQ ID NO: 22 - protein, 18 aa, Conus regius
  x (3)..(3): gamma-hydroxyproline
RDXCCYHPTCNMSNPQIC

SEQ ID NO: 23 - protein, 17 aa, Conus regius
  x (4)..(15): gamma-hydroxyproline
GCCXXQWCGXGCTSXCC

SEQ ID NO: 24 - protein, 17 aa, Conus regius
  x (4)..(5): gamma-hydroxyproline
LCCXXQXCGXDCASXCC

SEQ ID NO: 25 - protein, 15 aa, Conus regius
CCTALCSRYHCLPCC

SEQ ID NO: 26 - protein, 15 aa, Conus regius
  x (11)..(11): gamma-hydroxyproline
KCCMRPICTCXCCIG

SEQ ID NO: 27 - protein, 17 aa, Conus regius
GCCPFPACTHHIICRCC

SEQ ID NO: 28 - protein, 19 aa, Conus regius
  x (5)..(18): gamma-hydroxyproline
GCCSXWNCIQLRACXCCXN

SEQ ID NO: 29 - protein, 13 aa, Conus regius
  x (11)..(11): gamma-hydroxyproline
KCCMRPICMCXCC

SEQ ID NO: 30 - protein, 17 aa, Conus regius
RCCPMPGCFAGPFCPCC

SEQ ID NO: 31 - protein, 17 aa, Conus regius
  x (14)..(14): gamma-hydroxyproline
GCCGSFACRFGCVXCCV

SEQ ID NO: 32 - protein, 25 aa, Conus regius
  x (2)..(22): gamma-hydroxyproline
LXSCCSLNNLRLCXVXACKRNXCCT

SEQ ID NO: 33 - protein, 22 aa, Conus regius
  x (6)..(17): gamma-hydroxyproline
RDCCTXXKKCKDRQCKXQRCCA

SEQ ID NO: 34 - protein, 21 aa, Conus geographus
  x (7)..(17): gamma-hydroxyproline
RLCCGFXKSCRSRQCKXHRCC

SEQ ID NO: 35 - protein, 21 aa, C. purpurascens
  x (7)..(17): gamma-hydroxyproline
RLCCGFXKSCRSRQCKXHRCC

SEQ ID NO: 36 - protein, 25 aa, C. purpurascens
  x (2)..(13): gamma-hydroxyproline
HXCCLYGKCRRYXGCSSASASCCQR

SEQ ID NO: 37 - protein, 20 aa, Conus regius
  x (4)..(4): gamma-hydroxyproline
RVLXGXDXDVGXAGYHHLLX

SEQ ID NO: 38 - protein, 11 aa, Conus geographus
GEQNQLIRKSN

SEQ ID NO: 39 - protein, 16 aa, Conus tulipa
GEQKMLNLRAEVKKNA

SEQ ID NO: 40 - protein, 22 aa, Conus radiatus
GEAKMAALARNIAKGCKVNCYP

SEQ ID NO: 41 - protein, 14 aa, Conus lynceus
GEAKMAALARDAVN

SEQ ID NO: 42 - protein, 19 aa, Conus sulcatus
GDSKFIRERAGRLDLSKFP

SEQ ID NO: 43 - protein, 12 aa, Conus quercinus
GYRVATVRLDAA

SEQ ID NO: 44 - protein, 12 aa, Conus caracteristicus
GYRIATVRLEEA

SEQ ID NO: 45 - protein, 15 aa, Conus gloriamaris
GAKRNNAAVRRLEEI

SEQ ID NO: 46 - protein, 18 aa, Conus orchroleucus
GERKAMALEAKKAQALKA

SEQ ID NO: 47 - protein, 8 aa, Conus mus
  x (2)..(2): gamma-hydroxyproline
SXANSXWS

SEQ ID NO: 48 - protein, 8 aa, Conus mus
  x (2)..(2): hydroxyproline
SXANSVWS

SEQ ID NO: 49 - protein, 8 aa, Conus mus
  x (2)..(2): hydroxyproline
SXANSVWS

SEQ ID NO: 50 - protein, 8 aa, Conus gladiator
  misc_feature (2)..(2): Xaa can be any naturally occurring amino acid
AXANSXWS

SEQ ID NO: 51 - protein, 8 aa, Conus gladiator
  misc_feature (2)..(2): Xaa can be any naturally occurring amino acid
AXANSXWS

SEQ ID NO: 52 - protein, 8 aa, Conus gladiator
  x (2)..(2): hydroxyproline
AXANSVWS

SEQ ID NO: 53 - protein, 7 aa, Conus vilepinii
  x (2)..(2): hydroxyproline
EXNSMWS

SEQ ID NO: 54 - protein, 7 aa, Conus vilepinii
  x (2)..(2): hydroxyproline
EXNSIWS

SEQ ID NO: 55 - protein, 7 aa, Conus vilepinii
EPNSIWS